(19) United States
(12) Patent Application Publication
(10) Pub. No.: US 2022/0000341 A1
(43) Pub. Date: Jan. 6, 2022

(54) SINGLE USE DEVICES WITH INTEGRATED VISION CAPABILITIES

(71) Applicant: Noah Medical Corporation, Redwood City, CA (US)

(72) Inventor: Jian Zhang, Redwood City, CA (US)

(21) Appl. No.: 17/381,105

(22) Filed: Jul. 20, 2021

Related U.S. Application Data
(63) Continuation of application No. PCT/US2020/014959, filed on Jan. 24, 2020.
(60) Provisional application No. 62/796,477, filed on Jan. 24, 2019.

(51) Int. Cl. A61B 1/00; A61B 1/05; A61B 17/32; A61B 1/005; A61B 34/30

(52) U.S. Cl. CPC A61B 1/00103; A61B 1/05; A61B 1/00087; A61B 2017/0023; A61B 1/005; A61B 34/30; A61B 2017/320024; A61B 17/32002

(57) ABSTRACT

An integrated single use device with vision capabilities is provided. The device may comprise: an endoscope comprising: i) a disposable elongate member comprising a proximal end and a distal end and ii) a camera module located at the distal end, and the proximal end is removably attached to a supporting member; and one or more disposable instruments integrated to the endoscope, and the device is configured to perform functions of both the endoscope and the one or more disposable instruments.

### REFERENCE

This application is a continuation application of International Application No. PCT/US2020/014959, filed Jan. 24, 2020, which claims the benefit of U.S. Provisional Application No. 62/796,477, filed Jan. 24, 2019, each of which is incorporated herein by reference in its entirety.

### BACKGROUND OF THE INVENTION

Endoscopes comprise vast applications in diagnosis and treatment of various conditions, such as medical conditions. In many cases, endoscopes may be re-usable and may require cleaning procedures, sterilization, and disinfection which may be harsh to the endoscope and complicate the endoscope design and the procedures in which such endoscopes are used for.

Recognized herein are devices and systems comprising endoscopes which may be disposable and may not require extensive cleaning procedures. In addition, recognized is a need for devices comprising endoscopes and disposable instruments which may be integrated with the endoscope and may be configured to perform the functions of both the endoscope and the instruments as a single unit. Provided herein are different variations of such devices, as well as methods of use of such devices.

### SUMMARY OF THE INVENTION

Recognized herein is a need for systems and methods that allow for performing surgical procedures or diagnostic operations with improved reliability and cost-efficiency. Systems and methods may be minimally invasive.

In an aspect the present disclosure provides a device comprising: (a) an endoscope comprising: i) a disposable elongate member comprising a proximal end and a distal end and ii) a camera module located at the distal end, while the proximal end may be removably attached to a supporting member; and (b) one or more disposable instruments integrated to the endoscope. The device may be configured to perform functions of both the endoscope and the one or more disposable instruments.

In some embodiments, the distal end is articulated in two or more degrees of freedom. In some embodiments, at least one of the one or more disposable instruments may be integrated to the endoscope through a permanent connection at the distal end. In some embodiments, one or more pull wires may be integrated into the walls of the elongate member.

In some embodiments, the one or more disposable instruments are one or more instruments selected from the group consisting of a morcellator, scissors, a needle, an illumination source, a laser, a balloon, an implant/stent delivery device, a stitching device, laser fibers, a grasper, snares, a loop, and a bucket. In some embodiments, the one or more disposable instruments may further comprise other instruments.

In some embodiments, there may be substantially no relative movement between the camera module and the one or more disposable instruments. In some embodiments, the camera module and the one or more disposable instruments may be permanently integrated to the endoscope, and there may be substantially no relative movement between the camera module and the additional disposable instrument.

In some embodiments, the camera module may be configured to provide a stationary view of the one or more disposable instruments. In some embodiments, the camera module may be fixed relative to the one or more disposable instruments.

In some embodiments, the distal end comprises an endoscope function and an instrument function.

In some embodiments, the supporting member may comprise a mechanical interface configured to control an articulation of the distal end of the endoscope or operations of the one or more instruments. In some embodiments, the mechanical interface may comprise a driving mechanism for one or more pull wires attached to the distal end. In some embodiments, the mechanical interface may comprise a mechanism for manipulating the one or more disposable instruments.

In some embodiments, the device may further comprise an interface between the distal portion and the supporting member. In some embodiments, the supporting member may comprise or be a robotic arm. Alternatively, the supporting member may be coupled to a robotic arm. In some embodiments, the supporting member may be re-usable. In some embodiments, the supporting member may be configured to control functions of the endoscope and the one or more instruments.

In some embodiments, the supporting member may be a hand-held device. The hand-held device may comprise or be or be referred to as a hand piece.

In some embodiments, the endoscope may be disposable. In some embodiments, the one or more disposable instruments may comprise a morcellator. In some embodiments, the morcellator may be located at the distal end. In some embodiments, the camera module may reside next to the morcellator.

In some embodiments, the one or more disposable instruments may comprise an illumination source. In some embodiments, the illumination source may reside next to the camera. In some embodiments, the illumination source may comprise illumination fibers or one or more Light Emitting Diodes (LED).

In some embodiments, the endoscope may comprise a channel. In some embodiments, the morcellator comprises an outer tubing and an inner tubing. In some embodiments, there may be substantially zero relative motion between the morcellator and the camera module. In some embodiments, the device may be configured to provide a stationary view of the morcellator. In some embodiments, the morcellator is pre-bent. In some embodiments, the camera module may reside at the neck area of the morcellator. In some embodiments, the camera module may reside at other areas. In some embodiments, the morcellator may comprise a shaft. In some embodiments, the camera module and the morcellator may each comprise a shaft. In some embodiments, the shaft or shafts may be articulated in two or more degrees of freedom. In some embodiments, the shaft may comprise one or more pull wires.

In some embodiments, the one or more disposable instruments may comprise an illumination source and a morcellator. In some embodiments, the illumination source may be located within proximity to the camera module. In some embodiments, the illumination source may reside next to the camera module.

In some embodiments, the one or more disposable instruments may comprise an inflatable tip. In some embodiments, the inflatable tip may comprise an expandable balloon. In some embodiments, at least one of the one or more disposable instruments may comprise a shaft and a distal portion of the shaft may be connected to the distal end. In some embodiments, the inflatable tip may comprise at least one of a structural tube and an internal stiffener. In some embodiments, the device may be configured to provide a stationary view of the inflatable tip. In some embodiments, there is substantially no relative movement between the inflatable tip and the camera module.

In some embodiments, the one or more disposable instruments may comprise an implant/stent delivery device. In some embodiments, the implant/stent delivery device may be configured to perform sinus procedures. In some embodiments, the device may comprise an implant/stent delivery device and an illumination source. In some embodiments, the device may further comprise a channel. In some embodiments, the device may further comprise an implant/stent delivery pushing mechanism. In some embodiments, the implant delivery device may be configured to deliver a prostate implant.

In an aspect, the disclosure provides a method comprising: providing an endoscope comprising a disposable elongate member comprising a proximal end and a distal end, and a camera module located at the distal end, while the proximal end may be removably attached to a supporting member; providing one or more disposable instruments; and integrating the one or more disposable instruments to the endoscope to form an integrated single device.

In some embodiments, the method may further comprise performing functions of the endoscope and the one or more disposable instruments using the integrated single device.

In some embodiments, integrating the one or more disposable instruments to the endoscope to form the integrated single device may not require user-assembly. In some embodiments, the integrated single device may be provided in a sterilized packaging. In some embodiments, the method may further comprise selecting at least one disposable instrument from the one or more disposable instruments to be integrated prior to performing the functions.

In some embodiments, the one or more disposable instruments may be integrated to the endoscope using an adhesive material. In some embodiments, the adhesive material may be biocompatible. In some embodiments, the one or more disposable instruments may be integrated to the endoscope via one or more mechanical connection features. In some embodiments, the one or more mechanical connection features may comprise a quick-release mechanism. In some embodiments, the one or more mechanical connection features may comprise a snap-on mechanism. In some embodiments, the method may not require post-procedure cleaning of the device. In some embodiments, integrating the one or more disposable instruments to the endoscope comprise using one or more locks.

In some embodiments, integrating the one or more disposable instruments to the endoscope may comprise using a mechanism or tool selected from the group consisting of heat, welding, glue, friction, snap, lock, clip, rail, mechanical deformation, bonding with an adhesive epoxy, glue, biocompatible glue, curing with light, brazing, soldering, mechanical fastening, interlocking connections, and flanges.

In an aspect, the present disclosure provides an integrated disposable (or single-use) device with at least one integrated disposable instrument. The device may comprise vision capabilities provided by a camera module (such as a disposable camera module at the distal end of a disposable endoscope). In some embodiments, the device may be entirely disposable. The device may comprise a disposable endoscope comprising a distal end and a proximal end and a camera module at the distal end. The proximal end of the endoscope may be configured to be connected to a supporting member such as a hand-held device or a robotic arm. The endoscope and the camera module may be disposable. One or more disposable instruments may be integrated to the endoscope which may be configured to perform desired procedures (such as surgical procedures inside the body of a subject) while the endoscope provides a view of the surgical site and the instrument through the camera module. In some embodiments, the endoscope, the camera module, and the instruments may all be disposable and may be integrated together, thereby making a combined (or integrated) single device which may be configured to perform the functions of the endoscope and the one or more disposable instruments and may be entirely disposable. The disclosure further provides method of making and using such devices for various applications (such as medical and/or surgical applications). The device may be disposable, therefore may eliminate the need for post-procedure cleaning, and may simplify the endoscope design. The devices and methods provided herein may have advantages such as sterility, lowered cost, smaller overall size of the device compared to a case where the instruments and endoscope are not integrated into a single device, improved patient comfort, better usability, and simpler and more streamlined workflow among other benefits.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 schematically illustrate an example device 100 in accordance with some embodiments of the present disclosure.

FIGS. 2A and 2B shows an example of an alternative distal portion of a disposable integrated device with integrated camera module.

FIG. 3 shows an example of the proximal portion of a disposable integrated device with a camera module.

FIG. 4A is a device which can be used to deliver an implant into prostate.

FIG. 4B is a device which can be used to deliver high temperature water steam into prostate.

FIG. 5A is a device which can be used to deliver an implant within the cannel of the prostate.

FIG. 5B is a device which can be used to deliver a different type of implant within the cannel of the prostate.

FIG. 6A and FIG. 6B show examples of the proximal end and supporting member which may comprise a button, electrical interface, mechanical interface, fluid management interface, and other parts and pieces on the supporting member.

FIG. 7 shows an alternative example of the device shown in FIG. 1.

FIG. 8 shows an example of a combined device architecture comprising endoscope, camera, and components for controlling the instrument.

FIG. 9 shows examples of a device with integrated morcellator.

FIG. 10 shows examples of a device with integrated inflatable tip.

FIG. 11 shows examples of the combined implants/stents delivery devices.

FIG. 12 shows examples of the combined stitching devices.

FIG. 13 shows examples of the combined instruments.

FIGS. 14A-14E shows examples of the proximal end design of the integrated device and the supporting member.

FIG. 15 shows an example of the distal portion of a disposable integrated device with vision capabilities. The camera module resides in a channel of the disposable device.

FIGS. 16A and 16B shows an example of a distal portion of a disposable integrated device with vision which can be used to morcellate tissues under direct vision.

FIG. 17A is an example of a distal portion of a disposable device with integrated vision which can be used to deliver electrical cautery to tissue.

FIG. 17B is an example of a distal portion of a disposable or single-use device with integrated vision (or integrated camera module) which can be used to grasp tissue or delivery IUD.

FIG. 18 shows an example robotic arm according to the devices and methods of the present disclosure.

### DETAILED DESCRIPTION OF THE INVENTION

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU) or a microcontroller), digital signal processors (DSPs), a field programmable gate array (FPGA) and/or one or more Advanced RISC Machine (ARM) processors. In some cases, the one or more processors may be operatively coupled to a non-transitory computer readable medium. The non-transitory computer readable medium can store logic, code, and/or program instructions executable by the one or more processors unit for performing one or more steps. The non-transitory computer readable medium can include one or more memory units (e.g., removable media or external storage such as an SD card or random access memory (RAM)). One or more methods or operations disclosed herein can be implemented in hardware components or combinations of hardware and software such as, for example, ASICs, special purpose computers, or general purpose computers.

As used herein, the terms distal and proximal may generally refer to locations referenced from the apparatus, and can be opposite of anatomical references. For example, a distal location of a bronchoscope or catheter may correspond to a proximal location of an elongate member of the patient, and a proximal location of the bronchoscope or catheter may correspond to a distal location of the elongate member of the patient.

Endoscopes are widely used to examine the inside of anatomies of subjects, such as animal and human subjects. Subjects may comprise animals such as pig, mouse, dogs, mammals, rodents, monkey, and other animals. Subjects may comprise human subjects, such as patients. The methods and systems provided herein may be used for diagnosing and or treating subjects and/or parts of subjects.

Endoscopes are traditionally made to be re-usable, which may require thorough cleaning, dis-infection, and/or sterilization after each procedure. In most cases, cleaning, dis-infection, and sterilization may be aggressive processes to kill germs and/or bacteria. Such procedures may also be harsh on the endoscopes themselves. Therefore, the designs of such re-usable endoscopes can often be complicated, especially to ensure that the endoscopes can survive such harsh cleaning, dis-infection, and sterilization protocols. Periodical maintenance and repairs for such re-usable endoscopes may often be needed.

Many traditional endoscope designs may be complicated in part because the sterility requirement of endoscopes for each procedure may require the endoscope or endoscope parts to be washed, sterilized, and/or disinfected after and/or before each procedure. Endoscopes may be re-processed after each procedure. The re-processing may comprise washing cleaning, re-processing, sterilizing, and/or disinfecting after each procedure. In many cases, these procedures may be aggressive processes to kill germs and/or bacteria, and therefore, may be harsh on the endoscopes.

Recognized herein is a need for disposable or single-use devices comprising endoscopes (e.g., disposable or single-use endoscope). There are several advantages for having disposable devices. Disposable devices, such as disposable endoscopes may not require post-procedure cleaning, disinfection, and sterilization. Disposable or single-use devices may be disposed by the user after the procedure and save the user valuable time and effort. Additionally, since cleaning procedures may be harsh to the endoscopes and may oblige complex endoscope designs to be compliant with such cleaning protocols, having disposable endoscopes may simplify endoscope designs. Provided herein are devices comprising disposable endoscopes.

Moreover, oftentimes, instruments are used alongside or in combination with endoscopes which may require the user (e.g., surgeon) to hold an endoscope and one or more instruments or tools separately. Alternatively, multiple people may need to hold such instruments and endoscope separately. More space may be needed for accomplishing such procedure. Recognized herein is a need for providing an integrated device comprising an endoscope and one or more instruments which may be used in combination with it to save space and make the procedure easier and more convenient.

In some cases, instruments and devices that may be used in combination with endoscopes may be single-use or disposable. Such instruments or tools (e.g., disposable instruments) may comprise mechanical tools, lasers, inflatable tips (e.g., balloons), morcellators, implant/stent delivery devices, stitching devices, and other devices and instruments. These instruments may often be designed by a separate entity than the endoscope manufacturer. This may in part be due to the fact that in most cases, the endoscope may be complicated and difficult to design and manufacture, and the instrument manufacturers may choose to focus on designing instruments that work with a specific type of endoscope. Although disposable or single-use devices and instruments have become increasingly popular in recent years, there is a need for design and manufacturing disposable single-use endoscopes. As such, the majority if not all the currently available endoscopes are still re-usable. Provided herein are devices comprising disposable or single-use endoscope systems and uses thereof to address the shortcomings associated with re-usable endoscopes. In some examples, the single-use endoscopes may further comprise additional instruments (e.g., one or more disposable instruments) integrated in them as a single device. In some cases, such instruments may be provided separately from the single-use endoscopes. In many cases, it may be preferred to integrate such instruments (e.g., one or more disposable instruments) with the endoscope as a single unit/device due to the advantages it may provide which may comprise a small size of the entire device compared to a case where devices would be used separately, and convenience among other benefits.

In common endoscopy diagnosis (e.g., existing devices and methods), biopsies, and treatments, there may oftentimes be a separate instrument or several separate instruments to be used in combination with one or more endoscopes. This has been the medical practice for the past decades, at least partially because the endoscopes and the instruments may often be made at different times by different device manufacturers. Even when they are made and manufactured by the same entity, due to the cost of the endoscopes, which can be every expensive, capital equipment, instruments may in some cases be provided as separate devices from the endoscopes. These instruments can be of many different formats: Simple mechanical tools such as graspers, scissors, baskets, snares, curette, and other devices. Advanced instruments such as laser fibers, stitching tools, balloons, morcellators, various implant or stent delivery devices, and more. Such instruments can be used in many different endoscopy procedures in combination with many different kinds of endoscopes such as, NeuroendoScope, EncephaloScope, OphthalmoScope, OtoScope, RhinoScope, LaryngoScope, GastroScope, EsophagoScope, BronchoScope, ThoracoScope, PleuroScope, AngioScope, MediastinoScope, NephroScope, GastroScope, DuodenoScope, CholeodoScope, CholangioScope, LaparoScope, AmioScope, UreteroScope, HysteroScope, CystoScope, ProctoScope, ColonoScope, ArthroScope, SialendoScope, and more.

For example, morcellators can be used in gynecology and rhinology to treat polyps. Balloons can be used to do sinuplasty in rhinology. Some implants or stents can be used to treat fluid behind the ear drums (tympanostomy), open the sinus ostium, or treat benign prostatic hyperplasia (BPH) by opening the cannel at the prostate. A stitching mechanism can be used to do suture inside the stomach. Laser fibers can be used to do ablation or coagulation in urology, gynecology, etc.

Traditional endoscopes can be large in size (e.g., a few millimeters to a few centimeters in outside diameter). In order to accommodate any of the above-mentioned instruments, a working channel may be needed in the endoscope to allow for the instrument to pass through. In some cases, more than one working channel may exist to allow multiple instruments to pass through at the same time or at various time intervals. In most cases, this type of design may consider the instruments and the endoscopes to be two separate devices and therefore a clear mechanical interface may be defined between the instrument and the endoscope. Although this may simplify the designs of the instrument and the endoscope, in some cases, it may cause the overall size of the endoscopes to be bigger than necessary.

Recognized herein is a need for miniaturized endoscopes which may be low in cost and single-use. In some cases, the endoscopes provided herein may comprise integrated instruments such as vision instruments, cameras, sensors, illumination devices, and more. Such endoscopes may be used for treatments in various applications such as diagnosis and/or treatment of disease. The devices and methods may comprise applications for bronchoscopy, urology, gynecology, arthroscopy, ENT, gastro-intestine endoscopy, etc. In some cases, the devices provided herein may be miniaturized combined endoscopes (e.g., combined with other devices such as camera modules, illumination, and mode) and may be single-use to avoid the challenge of cleaning and disinfection. Key requirements to minimize the size (i.e. outer diameter) and keeping the cost low may be achieved by the imaging system design, illumination method and design, and the shaft design including articulation. Some of these objectives will be satisfied by devices described in the present disclosure.

Provided herein are disposable endoscopes. In some examples, provided are disposable single-use endoscopes with integrated instruments (e.g., disposable instruments). In some cases, the entire device is disposable or single-use. In some examples, provided is a device comprising an endoscope and one or more instruments integrated together in a single device. The device may comprise a camera module. The camera module and/or imaging sensors may be low in cost and/or disposable (or single-use). Material used may comprise specific qualities and features that may make them ideal to be incorporated in single-use/disposable endoscopes.

Among other advantages, this integration may favorably reduce, minimize, or optimize the overall size of the combined device. In some cases, the combined/integrated devices may be designed to be disposable or single-use, which may greatly reduce the chance of infection and decrease the post-processing requirements such as cleaning, disinfection, sterilization, etc.

In some examples, the devices and systems provided herein may have applications comprising the diagnosis and/or treatments of conditions (e.g., medical conditions) in subjects (e.g., humans or animals) such as conditions related to urology, gynecology, rhinology, otology, laryngoscopy, gastroenterology, and other areas. Applications may comprise addressing conditions in other parts of the subjects, such as eye, stomach, intestine, ovary, or more.

In some cases, the methods and systems provided herein can be combined in one or more ways to provide improved diagnosis and/or therapy to a subject (e.g., a patient). For example, the methods and systems can be combined with existing methods and apparatus to provide improved treatment or diagnosis, such as in combination with known methods of urological, gynecological, laryngological, ontological, gastroenterological diagnosis or surgery of other tissues and organs, for example. Similarly, the devices and/or methods provided herein may be combined with same or other instruments and/or tools to address, diagnose or treat other conditions, either healthy, diseases, benign or malignant in any human or non-human subject. It is to be understood that any one or more of the structures and/or procedures as described herein can be combined with any one or more additional structures and/or procedures of the methods and apparatus as described herein.

Provided herein are systems/devices and methods comprising an endoscope with integrated instruments/tools and capabilities for diagnosis and treatment of subjects. In some examples, endoscopes may be disposable or single-use endoscopes. The devices comprising disposable or single-use endoscopes may further comprise vision capabilities such as camera modules integrated in them. In some cases, the camera module may be integrated at the distal tip of the endoscope. Alternatively, the camera module may be at other locations of the device.

The device may further comprise one or more instruments. In some examples, the one or more instruments may be one or more disposable or single-use instruments. The instruments which may be used in combination with the endoscopes either separately or as an integral part of a single system or device comprising an endoscope (e.g., a single-use endoscope) may comprise mechanical tools, electrical tools, optical tools, sensors, cameras or camera modules, chemicals, materials (e.g., degradable materials, biocompatible materials, hydrogels, etc.), medications, lasers, inflatable tips (e.g., balloons), morcellators, implant/stent delivery devices, stitching devices, and more, and combinations thereof, in any configuration and/or setting (e.g., built-in, integrated, or used separately).

In some examples, other tools and/or instruments may be used in combinations with the devices provided herein either separately or integral within the same device or system. Such instruments may comprise NeuroendoScope, EncephaloScope, OphthalmoScope, OtoScope, RhinoScope, LaryngoScope, GastroScope, EsophagoScope, BronchoScope, ThoracoScope, PleuroScope, AngioScope, MediastinoScope, NephroScope, GastroScope, DuodenoScope, CholeodoScope, CholangioScope, LaparoScope, AmioScope, UreteroScope, HysteroScope, CystoScope, ProctoScope, ColonoScope, ArthroScope, SialendoScope, other tools and instruments, and/or any combination thereof in any configuration and/or setting (e.g., built-in, integrated, or used separately).

In some examples, the present disclosure provides a method comprising: providing an endoscope comprising a disposable elongate member comprising a proximal end and a distal end, and a camera module. located at the distal end, while the proximal end may be removably attached to a supporting member; providing one or more disposable instruments; and integrating the one or more disposable instruments to the endoscope to form an integrated single device. In some examples, the camera module may be in locations other than the distal end. For example, the camera module may be on a separate shaft in the device.

In some examples, the method may further comprise performing functions of the endoscope and the one or more disposable instruments using the integrated single device.

In some examples, integrating the one or more disposable instruments to the endoscope to form the integrated single device may not require user-assembly. In some embodiments, the integrated single device may be provided in a sterilized packaging. In some embodiments, the method may further comprise selecting at least one disposable instrument from the one or more disposable instruments to be integrated prior to performing the functions. For example, the endoscope, the camera module, and the one or more instruments (e.g., disposable instruments) may be provided to a user. The user may select a combination of endoscope, camera module, and/or instruments to use. The user may assemble and use the device. Alternatively, in some examples, the endoscope, camera module and the one or more instruments (e.g., disposable instruments) may be assembled before being provided to the user. For example, the parts may be permanently attached, connected or otherwise integrated using any methods or mechanisms provided herein and be ready-to-use without any need for user assembly. Additionally, the device may be disposable. So, the user may not need to either assemble the device or perform post-procedure cleaning. This may provide significant advantage to the user. In addition, since post-procedure cleaning is not needed, endoscope may not need to be designed to be compatible with such post-procedure cleaning protocols which are oftentimes harsh to the endoscope. Therefore, the endoscope design and/or manufacture may be simplified.

In some examples, the one or more instruments (e.g., disposable instruments) may be integrated in the device (e.g., to the endoscope) using an adhesive material. In some examples, the adhesive material may be biocompatible. In some examples, the one or more instruments (e.g., disposable instruments) may be integrated to the device (e.g., to the endoscope) via one or more mechanical connection features. In some examples, the one or more mechanical connection features may comprise a quick-release mechanism. In some examples, the one or more mechanical connection features may comprise a snap-on mechanism. In some examples, the device may be integrated and packed (e.g., in a sterile packaging) and may be further delivered to the user. Therefore, the user may not be required to assemble the device before the procedure.

In some examples, the device may be disposable. Hence, the method may not require post-procedure cleaning of the device. In some examples, integrating the one or more instruments (e.g., disposable instruments) in the device (such as to the endoscope) may comprise using one or more locks.

In some examples, integrating the one or more instruments (e.g., disposable instruments) in/to the device (e.g., to the endoscope) may comprise using a mechanism or tool selected from heat, welding, glue, friction, snap, lock, clip, rail, mechanical deformation, bonding with an adhesive epoxy, glue, biocompatible glue, curing with light, brazing, soldering, mechanical fastening, interlocking connections, flanges, and/or other instruments, tools, and/or mechanisms or any combination thereof.

Provided herein are devices and methods for treatment of conditions in subjects or parts of subjects. Conditions may comprise healthy or diseased conditions, such as benign prostatic hyperplasia (BPH) or other human or animal diseases. Subjects may comprise animal subjects, such as mouse, pig, horse, monkey, rats, mammals, rodents, and other animals. Subjects may comprise human subjects, such as patients. Patients may be healthy or ill with different kinds of illnesses in different parts of their bodies. Parts of subjects may comprise sinuses, ureter, colon, esophagus, blood vessels, throat, brain, heart, lungs, intestines, eyes, skin, liver, pancreas, stomach, uterus, ovaries, testicles, bladder, ear, nose, mouth, bone marrow, adipose tissue, muscle, glandular, mucosal tissues, spinal, nerve tissues, cartilage, teeth, and bone, and other parts.

In some examples, the present disclosure provides methods and systems (such as single-use devices with integrated vision capabilities) for treating benign prostatic hyperplasia and other conditions. Benign prostatic hyperplasia (BPH) is a common disease for men, especially among older men. Minimally invasive surgical treatments to BPH are often used in practice. They include but are not limited to: transurethral resection of the prostate (TURP), transurethral incision of the prostate (TUIP), transurethral microwave thermotherapy (TUMT), transurethral needle ablation (TUNA), laser ablative procedures, photoselective vaporization of the prostate (PVP) and holmium laser ablation of the prostate (HoLAP), holmium laser enucleation of the prostate (HoLEP), prostate lift (i.e. Urolift), stent, or implant (i.e. Zenflow implant), water vapor therapy (i.e. Rezum).

In the above-mentioned minimally invasive procedures, a cystoscope or resectoscope is often used to gain access to the bladder, prostate, or urethra. These endoscopes are often comprised of multiple parts including sheath, telescope, tool channel and water channels. In many cases, they may require assembly of several or all pieces together before surgical procedure.

In some scenarios, the treatment tool or instrument may then be inserted into the working channel. For example, laser fiber may be inserted into the working channel to do PVP, HoLAP, or HoLEP. Unipolar or bipolar instruments with electrical loop may be inserted to do TURP. In some examples, one or more needle(s) may be inserted to do TUNA. In other scenarios, implant, stent or lift delivery instrument(s) may be complicated and have a dedicated working channel to insert a telescope. These instruments may have a built-in water channel as well. For example, Zenflow implant delivery device. Similarly, a steam delivery device may also include a channel to insert a telescope, i.e. Rezum device.

Often, the above-mentioned instruments are designed to be low-cost and single-use for sterility reasons. Endoscopes, however, can be very expensive due to precisely made optics and stay as a reusable piece and often used as an equipment. In some cases, prior to surgical procedures, physicians may need to assemble the reusable endoscopes into single use instruments or vice-versa. After most/all procedures, dis-assembly the endoscopes from instruments followed by cleaning and sterilization of the endoscopes may be necessary.

Traditionally, endoscopes may often include a working channel which may allow the instruments to go through and operate under endoscopy views. Alternatively, instruments may have scope channels that may allow telescopes to be inserted into, such that the instruments can operate under endoscopy views. With more commonly used single-use instruments, this combination makes a mixed scenario where endoscopes may still require cleaning and sterilization. Identified herein is a single-use package with single-use instrument and single-use endoscope. The systems, devices, and methods provided herein may improve the clinical workflow and efficiency.

When both instrument and endoscope are single-use, it may be desirable to integrate them into one combined device. With a low-cost camera module, integration of such cameras into single-use devices may be desirable to streamline the workflow of such devices. Integration of instruments with vision capabilities can optimize device overall size and minimal patient pain.

Traditionally, endoscopes usually require using a stack of optical lenses, which may make it more difficult to implement a flexible and soft endoscope. With small and low-cost camera modules, flexible and soft shafts may be desirable. It may be a major step forward to improve patient comfort in many applications, such as BPH treatment, and other treatments and procedures.

An integrated disposable (or single-use) device with integrated instrument and vision capabilities can provide advantages such as sterility, lowered cost, smaller overall sizes, improved patient comfort, better usability, simpler and more streamlined workflow among other benefits.

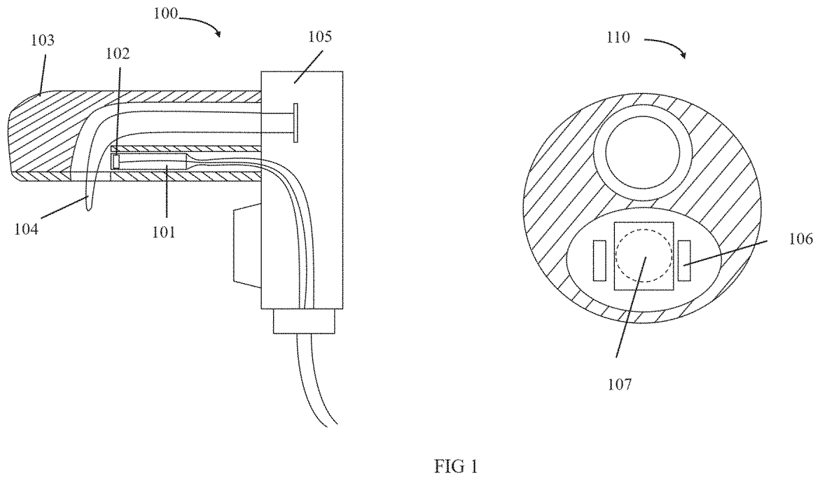

FIG. 1 schematically illustrate an example device 100 in accordance with some embodiments of the present disclosure. In other examples, the device may comprise other designs, configurations, and uses, such as other designs, configurations and uses provided elsewhere herein. In some cases, the device as illustrated in FIG. 1 can be used for various applications such as urological applications, i.e. BPH. The device can also be used to treat other parts of the body or other deceases, i.e. stone treatment. Examples include but not limited to: sinuses, ureter, colon, esophagus, blood vessels, throat, brain, heart, lungs, intestines, eyes, skin, liver, pancreas, stomach, uterus, ovaries, testicles, bladder, ear, nose, mouth, bone marrow, adipose tissue, muscle, glandular, mucosal tissues, spinal, nerve tissues, cartilage, teeth, and bone. Examples of some variations of devices are provided in other figures.

Referring to FIG. 1, the device 100 may be a disposable (single-use) integrated device. For example, the device may comprise a disposable endoscope with vision capabilities (such as a disposable endoscope comprising a camera module which may also be disposable). In some cases, the device may be used more than once or may comprise some parts which may not be disposable. Providing a device which is in part or in whole disposable (preferably entirely disposable) may have the advantage that the device may not need post-procedure cleaning and sterilization and may simplify endoscope design. In some cases, the device may comprise vision capabilities (such as a camera module). A camera sensor with lens 101, i.e. CMOS, CCD, or other types of sensors or camera modules may reside at distal of the device. The camera module (e.g. camera sensor) may be a low-cost camera sensor with lens. In some cases, the low-cost camera may be disposed after the procedure. In some examples, an illumination source (e.g., integrated light source) may be attached to the distal tip 102, i.e. LED, etc. In some examples, the camera module and the illumination source may be attached to the device distal tip. In some cases, the camera module and/or the illumination source may be attached permanently. Alternatively, the camera or the light source or both may be removably attached to the device distal tip. In other examples, the illumination (or light) source may be attached either permanently or removably to another location/zone on the device.

Referring to FIG. 1, in some examples, the camera module 101 can be tilted with a given angle inside the device (e.g., integrated device). For example, the given angle may be 0 degrees, 1 degree, 5 degrees, 10 degrees, 12 degrees, 18 degrees, 30 degrees, or other angles. This may create an endoscope with side view. In some cases, the camera module may not be tilted inside the device. In some examples, the camera module may be tilted at an angle of at least about 1 degree, 5 degrees, 10 degrees, 12 degrees, 15 degrees, 18 degrees, 20 degrees, 25 degrees, 30 degrees, 35 degrees, 40 degrees, or more. In some examples, the camera module may be tilted at an angle of at most about 60 degrees, 55 degrees, 50 degrees, 45 degrees, 40 degrees, 35 degrees, 30 degrees, 25 degrees, 20 degrees, 18 degrees, 12 degrees, 10 degrees, 5 degrees, 1 degree, or less.

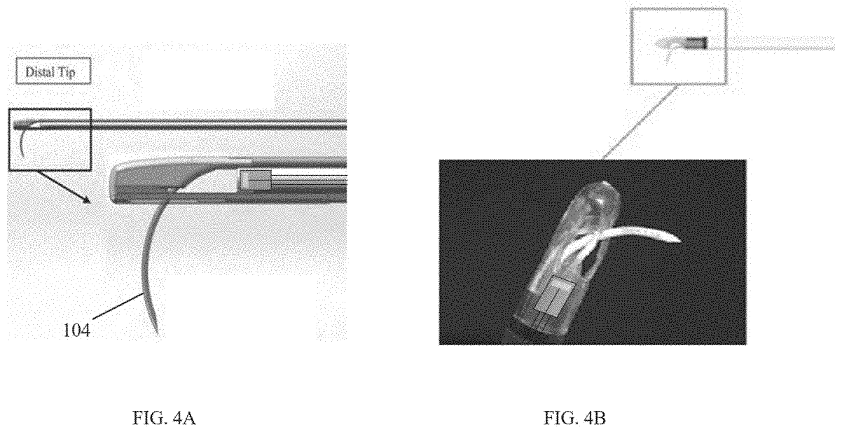

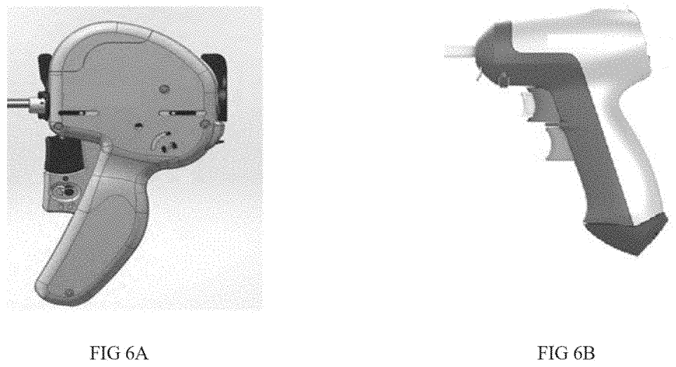

Referring to FIG. 1, in some examples, the device may comprise one or more shafts. In some cases, shafts may comprise a multi-lumen shaft. Referring to FIG. 1, Inside the device shaft 103 (e.g., multi-lumen shaft), one channel may be used to allow a semi-rigid, needle-like tube 104 to curve sideways and out of the shaft. Implants can be pushed through the needle-like tube into patient body using different methods, for example, by mechanisms inside a proximal hand-held device (or hand piece), such as the one illustrated in FIGS. 6A and 6B. As an example, the needle-like tube 104 may comprise or be an implant delivery channel, such as the channel shown in FIG. 4A.

Referring to FIG. 1, another use of the channel 104 may allow steam to go from proximal end to distal tip of the bent needle (shown in FIG. 4B). Steam may comprise any pressure and/or any temperature. In some cases, steam may comprise a high pressure and a high temperature. In some cases, the same channel may be used to deliver other media or instrument, i.e. medication, BOTOX® (onabotulinumtoxinA), green laser fiber, holmium laser fiber, or other media, instruments, and/or materials into subjects or parts of subjects. Alternatively, other channels in the device may be used to deliver such media, materials, and/or instruments into intended parts of subjects. For example, in some cases, the device may comprise multiple channels for delivering multiple materials or instruments. The channels may be similar to one another or may be different.

Referring to FIG. 1, in some cases, the channel can allow fluid to go in or out of the patient body. Another view 110 of the device is shown in FIG. 1. The cross-sectional view of the device 110 comprising the cross-sectional view of the camera module 107 and the cross-sectional view of one or more illumantion sources 106 are also shown in FIG. 1. Proximal end of the device is shown as a block in 105. Details of the proximal end of an example device provided herein can be found in FIG. 3. In other examples, the device may comprise other designs, configurations, and uses, such as other designs, configurations and uses provided elsewhere herein.

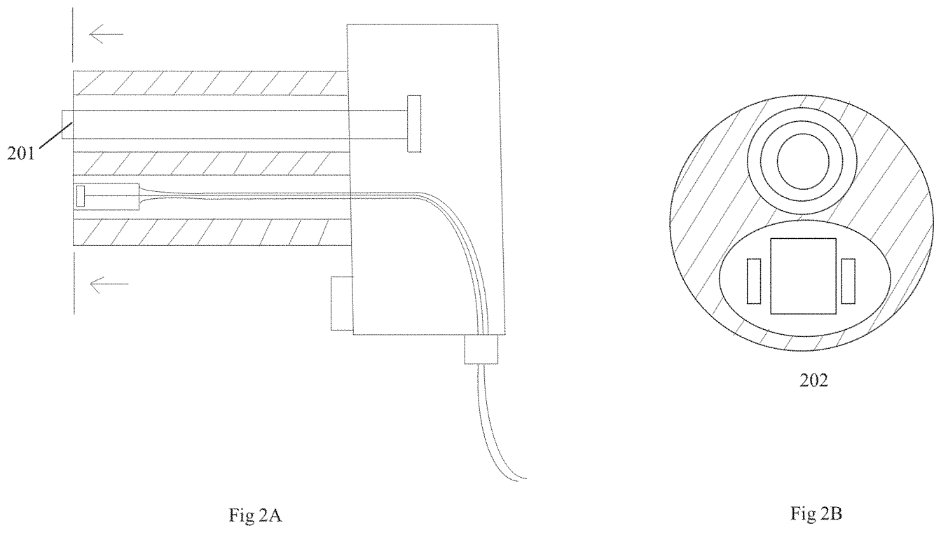

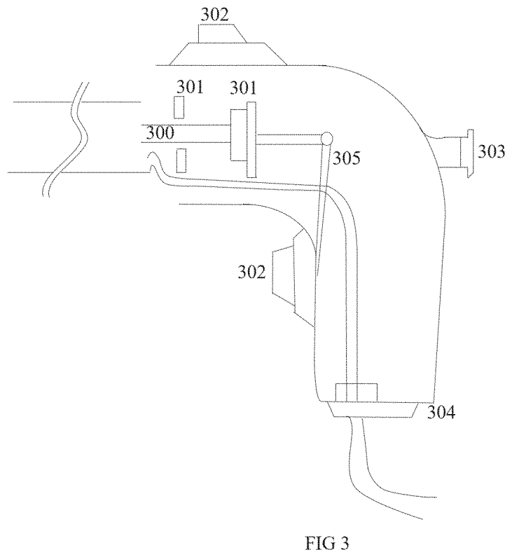

FIGS. 2A and 2B show an example device according to the devices and methods provided herein. The distal portion of an example device is shown in FIGS. 2A and 2B. The device may be a disposable (single-use device) with vision capabilities. For example, the device may comprise an endoscope comprising a camera module. The endoscope may be disposable. The camera module may be disposable. The device may be disposable in part or in whole. The device shown in FIGS. 2A and 2B may be an alternative design for the device shown in FIGS. 1A and 1B. Similar to the arrangement in FIG. 1, the camera module and illumination source may both be attached to the distal tip. The device shown in FIGS. 2A and 2B may further comprise one or more disposable instruments. For example, the one or more disposable instruments may comprise or be an implant/stent delivery device, apparatus, or instruments. The device may further comprise a channel 201. In some examples, the channel may be an implant/stent delivery channel 201 which may reside on the device (e.g., next to the camera module). The channel (e.g., delivery channel) may move relatively to the shaft of the device axially. A cross sectional view of the front face 202 is shown in FIG. 2B.

In some examples, the devices provided herein may comprise a camera module. In some examples, the devices may comprise one or more camera or imaging modules or systems. In some examples, the camera module may comprise a fluoroscopy (tomosynthesis) imaging system for providing real-time imaging of a target site (e.g., comprising a certain condition). In some cases, the camera module or imaging system may track the location of a given object inside the body or be used for verification purposes before or during surgical procedure. In some cases, the location or motion of the camera module (e.g., fluoroscopy (tomosynthesis) imaging system) may be measured using any suitable motion/location sensors such as inertial measurement units (IMUs)), one or more gyroscopes, velocity sensors, accelerometers, magnetometers, location sensors (e.g., global positioning system (GPS) sensors), vision sensors (e.g., imaging devices capable of detecting visible, infrared, or ultraviolet light, such as cameras), proximity or range sensors (e.g., ultrasonic sensors, lidar, time-of-flight or depth cameras), altitude sensors, attitude sensors (e.g., compasses) and/or field sensors (e.g., magnetometers, electromagnetic sensors, radio sensors). The one or more sensors for tracking the motion and location of the fluoroscopy (tomosynthesis) imaging station may be disposed on the imaging station or be located remotely from the imaging station, such as a wall-mounted camera. The various poses may be captured by the one or more sensors as described anywhere herein.

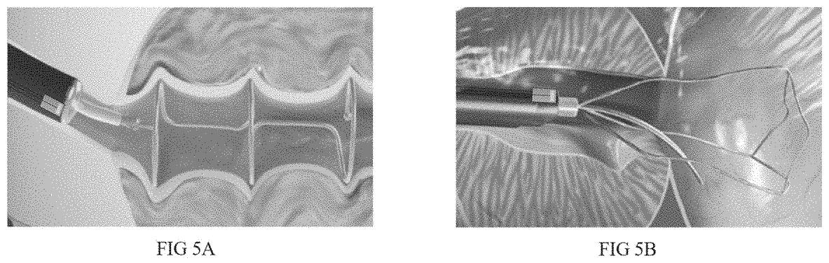

Two examples of the devices provided herein are shown in FIGS. 5A and 5B. In some examples, the device may comprise a camera module which may be next to a device channel, such as the implant delivery channel.

In some examples, the channel (such as channel 201 shown in FIG. 2A) can hold mechanical, electrical, optical, or other energy devices such as, graspers, scissors, snares, buckets, needles, uni-polar, bi-polar cautery instruments, RF ablation instrument, green laser fiber, holmium laser fiber, and/or other devices.

Referring to FIG. 2A, in some cases, the channel 201 can hold needles for biopsy, steam delivery, cryo therapy delivery, medication delivery, BOTOX, and/or other procedures.

In some examples, the devices shown in FIG. 1, FIG. 2A and FIG. 2B may comprise and/or use a rigid, semi-rigid, flexible, or articulatable device shaft. In such cases, the implant delivery channel may also be rigid, semi-rigid, flexible, or articulatable. In some examples, the distal end of the device may be articulated in two or more degrees of freedom.

The device or the endoscope may comprise a proximal end. The proximal end may be attached (e.g., removably attached) to a supporting member. In some examples, the supporting member may comprise or be a hand-held device.

In some examples, the supporting member may comprise a mechanical interface configured to control an articulation of the distal end of the endoscope or operations of the one or more instruments (e.g., disposable instruments). In some examples, the mechanical interface may comprise a driving mechanism for one or more pull wires attached to the distal end. In some examples, the mechanical interface may comprise a mechanism for manipulating the one or more disposable instruments.

One example of the proximal end of the device connected to a supporting member is shown in FIG. 3. In this example, the supporting member is a hand-held device (also referred to as a hand piece). Alternatively, in other examples, the supporting member may comprise or be a robotic arm or be configured to be attached or connected to a robotic arm. Two examples of the hand-held device (or hand piece) are shown in FIGS. 6A and 6B.

The supporting member, such as the hand-held device or the robotic arm may be configured to control or manipulate the functions of the device comprising the functions of the endoscope, the camera module, and/or the one or more disposable instruments. As an example, the one or more instruments (e.g., the one or more disposable instruments) may comprise or be an implant/stent delivery device. The device may further comprise a channel. The supporting member, such as the hand-held device (or hand piece) or a robotic arm may further comprise an interface for controlling, performing, and/or manipulating the functions of the endoscope and/or the one or more disposable instruments. In some examples, the interface may comprise or be a mechanical interface. In some examples, the interface may comprise or be an electrical interface. In some examples, the interface may comprise or be buttons. Buttons may comprise mechanical and/or electrical buttons. The channel may be used to deliver an implant to an intended part of a subject. In this example, the hand-held device (or hand piece) may be configured to control the functions of the endoscope and the implant delivery device.

Referring to FIG. 3, Inside the hand-held device (or hand piece), as an example, the channel (e.g., the working channel or the implant delivery channel) can have one or more hard stops 301. The channel may be an implant delivery channel, such as a channel which may be used to deliver an implant or stent into an intended part of a subject. In other examples, the channel may comprise other applications. The hard stops may be along the axial direction. This may provide safety stops to avoid too much insertion of the channel into the subject's body. In some examples, a mechanical and/or electrical interface (e.g., buttons 302) can reside on the proximal side. The mechanical and/or electrical buttons may enable delivery of implants/stents, and/or may be used for other purposes and/or applications. Other applications of the interface or buttons may comprise insertion of the channel (e.g., the delivery channel). In some cases, the device may comprise more buttons. The same or other buttons may allow image capture, video recording, white balance, and/or other functions.

Referring to FIG. 3, In some cases, one or more mechanism(s) may connect the mechanical and/or electrical buttons 302 to the implant delivery channel or implant cartridge. An example may comprise a hinge 305 that may connect the button 302 to the channel 300. In some cases, a different mechanism may be used and/or implemented. In some examples, such mechanisms may be complicated (e.g., more complicated than a hinge). Mechanisms may comprise or be mechanical and/or electrical mechanisms or combinations thereof. For example, an electrical interface may comprise a control system which may send signals (e.g., electrical or digital signals) to different parts of the device and thereby controlling the functions of the device, such as the endoscope, the supporting member, the one or more disposable instruments, or more.

Referring to FIG. 3, in some cases, the device or system may comprise one or more interfaces 303 for fluid management. Such interface(s) may be present at the proximal end of the device or other locations and/or zones in the device. In some cases, the interfaces may be lure connectors to enable inflow or outflow of fluid, steam or other media. In some cases, the interfaces may be used to allow implant delivery instruments or other instruments (e.g., instruments mentioned elsewhere herein such as the one or more disposable instruments) to go through. Another use of the interfaces may be to allow a separate endoscope (such as a single-use or disposable endoscope) to be inserted and snapped to the rest of the device. In some cases, this may be used when there is no integrated camera module at the distal tip of the device.

A mechanical and/or electrical interface 304 may be present to allow the device to connect to an image processing and/or display system, for example a laptop PC with a display monitor, a computing device, an iphone, ipad, tablet, or any other kind of computing device, machine or display system. Alternative options may comprise an imaging processing unit and a display monitor.

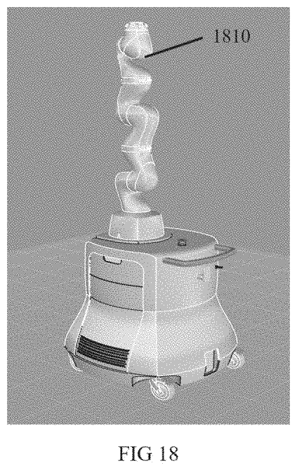

In some examples, the supporting member may be a robotic arm or be connected to a robotic arm. FIG. 18 shows an example of a robotic arm 1810 which may be used. In some examples, such as shown in FIG. 18, the robotic arm may be mounted on top of a robot cart. The robotic arm 1810 may automatically position intended parts of the device to an initial position (e.g., an access point) to access the target part of the subject (e.g., tissue). In some cases, the robot arm can be passively moved by an operator. In such case, an operator can push the arm in any position and the arm compliantly moves. In some cases, the robotic arm can be controlled in a compliant mode to improve human robot interaction. For example, the compliant motion control of the robot arm may employ a collision avoidance strategy and the position-force control may be designed to save unnecessary energy consumption while reducing impact of possible collisions. In some embodiments, the instrument driving mechanism may be mounted to the robotic arm. The arm may have redundant degrees of freedom allowing for its elbow to be algorithmically, or passively, moved into configurations that may be convenient for an operator.

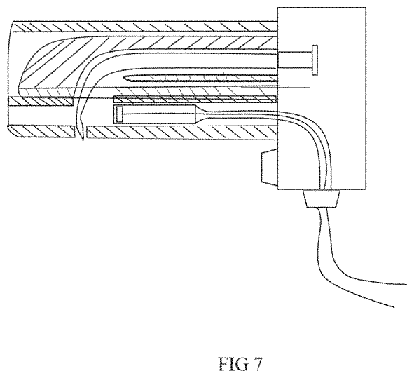

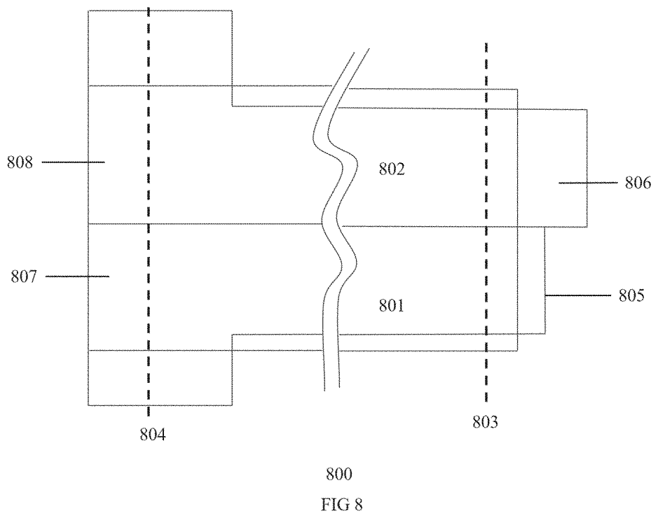

Provided herein is a device comprising an endoscope which may comprise an elongate member. The elongate member may comprise a proximal end and a distal end. The endoscope may be disposable. The device may further comprise a camera module. In some examples, the camera module may reside at the distal end of the endoscope. In other examples, the camera module may reside at other areas on the device. In some examples, the device may comprise one or more camera modules at different locations on the device. Referring to FIG. 7, For example, the device may comprise an outer shaft. The outer shaft may comprise a camera module (e.g., embedded in it). The outer shaft may further comprise an illumination source. In some examples, an implant/stent delivery shaft or a steam delivery shaft may reside inside another channel of the outer shaft. FIG. 7 shows one example of such design. Shaft(s) may be articulated. One or more pull wires may articulate the shaft(s).

In some cases, for design simplicity or adaptability reasons, camera module (e.g., a disposable, single-use camera module) with illumination can be on a separate shaft, which may get inserted into a channel inside the device shaft. Once inserted, the camera module shaft can be snapped onto the device shaft to make it one integrated device. In this example, in some cases, the camera module shaft may be removed from the integrated device.

The device may further comprise one or more instruments (e.g., disposable instruments) integrated thereon. In some cases, a disposable endoscope, a camera module, and one or more instruments (e.g., disposable instruments) may be integrated into a single device. The integrated single device may be disposable. In some examples, the camera module may be at the distal tip of the endoscope. In other examples, the camera module may reside at other areas of the device and may not be at the distal tip of the endoscope. For example, in the device shown in FIG. 7, the camera module may be embedded on a separate shaft in the integrated device.

In an aspect, the present disclosure provides a device comprising: (a) an endoscope comprising: i) a disposable elongate member comprising a proximal end and a distal end and ii) a camera module which may be located at the distal end. The proximal end of may be removably attached to a supporting member. The device may further comprise one or more instruments integrated to the endoscope. The instruments may be disposable or single-use. The device may be configured to perform functions of both the endoscope and the one or more disposable instruments.

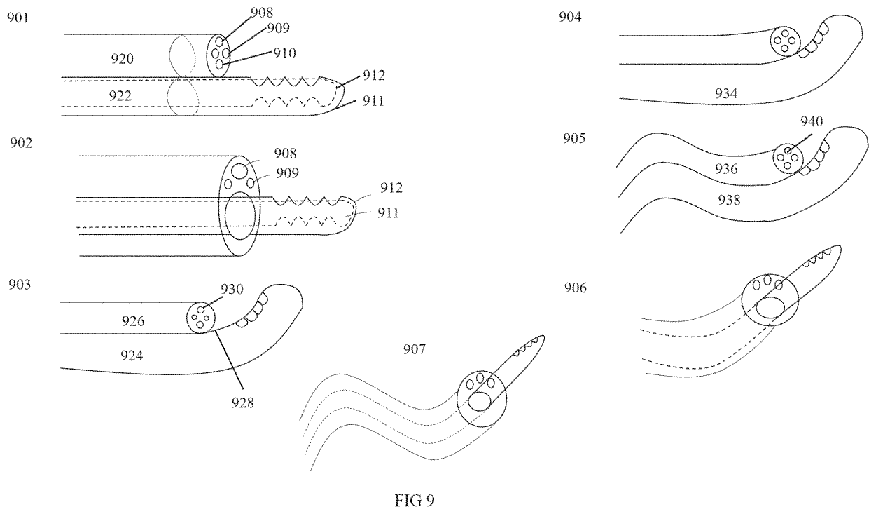

FIG. 8 shows one example device 800 according to the devices of the present disclosure. The device comprises an endoscope 801. The device may further comprise one or more instruments 802 (one instrument is shown as an example). The one or more instruments may be one or more disposable instruments. The device comprises a distal end 803 and a proximal end 804. The distal end of the device 803 may comprise an endoscope function and an instrument function. The endoscope may comprise an endoscope distal end 805 and an endoscope proximal end. The instrument may comprise an instrument distal end 806 and an instrument proximal end.

The endoscope 801 may comprise an elongate member. The elongate member of the endoscope may also be referred to as a shaft 103 or a catheter. An example of an endoscope comprising a shaft is shown in FIG. 1.

In some examples, the outer diameter of the distal tip of the endoscope (such as the diameter of the elongate member, shaft or catheter) may be around 4 to 4.4 millimeters (mm). In some examples, the endoscope may comprise a channel. The diameter of the channel may be around 2 mm.

In some examples, the endoscope may comprise a camera module. The camera module may be at the distal end of the endoscope. The camera module is not shown in FIG. 8. An example endoscope comprising a camera module 107 is shown in FIG. 1. Other examples of devices comprising camera modules are shown in FIG. 9 and other figures.

In some examples, at least one of the one or more disposable instruments may be integrated to the endoscope through a permanent connection at the distal end (such as at the distal end of the endoscope 805 or the distal end of the device 803).

In some examples, one or more pull wires may be integrated into the walls of the elongate member. Pull wires may facilitate articulation of the device or relevant parts of the device.

The mechanical packaging of the endoscope and the instrument can be in a variety of forms. The proximal end of the device 804 may comprise or be connected to a supporting member. The supporting member may comprise or be a hand-held device (or hand piece). An example of a hand-held device is shown in FIG. 3 and described in further detail in the following paragraphs.

In some examples, the supporting member may be a hand-held device. The hand-held device may comprise or be or be referred to as a hand piece. The hand piece may comprise functions of the instrument and the endoscope which may be combined into one (or the same) hand piece as an integrated device or system such as the example device shown in FIG. 8 or any other figure.

In some examples, the device may further comprise an interface between the distal portion and the supporting member. In some examples, the supporting member may comprise or be a robotic arm. Alternatively, the supporting member may be coupled to a robotic arm. In some embodiments, the supporting member may be re-usable. In some embodiments, the supporting member may be configured to control functions of the endoscope and the one or more instruments.

An example of a robotic arm is shown in FIG. 18 and described in further detail in the following paragraphs. The supporting member may be re-usable. The device comprising the endoscope and one or more instruments may be disposable.

In some cases, the instrument and the endoscope may be used in combination, but not integrated into the same device. For certain purposes, it may be desired to integrate the instrument and the endoscope.

Optionally, an interface may exist between the distal portion of the device (endoscope and instrument combined) and the supporting member (e.g., hand-held device, hand piece, or robotic arm). The interface is optional and if it exists, the distal portion of the device can be disposable and single-use and the supporting member (e.g., hand piece) can be designed to be re-usable.

Integrated Device with Endoscope and Morcellator

In some examples, the one or more instruments (e.g., disposable instruments) may be one or more instruments selected from a morcellator, scissors, a needle, an illumination source, a laser, a balloon, an implant/stent delivery device, a stitching device, laser fibers, a grasper, snares, a loop, a bucket, and/or any combination thereof. In some examples, the one or more disposable instruments may further comprise other instruments.

In some examples, the one or more instruments (the one or more disposable instruments) may comprise an illumination source and a morcellator. In some examples, the illumination source may be located within proximity to the camera module or next to the camera module. In some examples, the illumination source may reside next to the camera module.

In some examples, the endoscope may comprise a channel. In some examples, the diameter of the channel may be about 2 mm. In some examples, the diameter of the channel may be between 1 mm to 3 mm. In some examples, the morcellator may comprise an outer tubing and an inner tubing. In some examples, there may be substantially zero relative motion between the morcellator and the camera module. In some examples, the device may be configured to provide a stationary view of the morcellator. In some examples, the morcellator is pre-bent. In some examples, the camera module may reside at the neck area of the morcellator. In some examples, the camera module may reside at other areas. In some examples, the morcellator may comprise a shaft. In some examples, the camera module and the morcellator may each comprise a shaft. In some examples, the shaft or shafts may be articulated in two or more degrees of freedom. In some examples, the shaft may comprise one or more pull wires.

Referring to FIG. 9, In some examples, the device (e.g., device 901) may comprise an endoscope 920 (e.g., a disposable endoscope), a camera module (e.g., a low-cost disposable camera module at the distal tip of the endoscope or other locations), and one or more instruments (e.g., disposable instruments). The one or more instruments may comprise a morcellator.

Referring to FIG. 9, a camera 908 (such as a disposable camera) can be mounted next to the morcellator 922 at the distal end to form a combined device. This example may be referred to as a side-by-side configuration 901. The combined device may be a single unit which may be entirely disposable. Alternatively, it may not be entirely disposable and may comprise some re-usable pieces. The device may be partially or entirely disposable.

In some examples, the one or more instruments (e.g., the one or more disposable instruments) may comprise an illumination source.

Referring to FIG. 9, One or more illumination sources or apparatus 909 may reside on the device (e.g., device 901 or other example devices), for example, next to the camera 908. The illumination sources or apparatus may comprise or be illumination fibers, LEDs, or other illuminating parts, materials, or devices. The illumination source may be low-cost and disposable.

In some examples, the distal end of the endoscope and/or the instrument may be articulated in two or more degrees of freedom. Different variations and configurations of such devices are illustrated herein, such as in FIGS. 9-13, and 14A-E.

In some examples, the endoscope may optionally comprise a channel. In some examples, the diameter of the channel may be about 2 mm. In some examples, the diameter of the channel may be between 1 mm to 3 mm. In some examples, the morcellator may comprise an outer tubing and an inner tubing. In some examples, there may be substantially zero relative motion between the morcellator and the camera module (such as the camera module of the endoscope). In some examples, the device may be configured to provide a stationary view of the morcellator. In some examples, the morcellator is pre-bent. In some examples, the camera module may reside at the neck area of the morcellator. In some examples, the camera module may reside at other areas. In some examples, the morcellator may comprise a shaft. In some examples, the camera module and the morcellator may each comprise a shaft. In some examples, the shaft or shafts may be articulated in two or more degrees of freedom. In some examples, the shaft may comprise one or more pull wires.

Referring to FIG. 9, the device 901 (such as the endoscope of the device) may further comprise a channel 910. In some cases, the channel may be referred to as the working channel. In some examples, the channel may be an implant delivery channel. The channel may comprise other purposes and/or applications. The channel may reside at the distal end or at other places on the device. In some cases, the device may comprise a morcellator and the channel can be shared with the morcellator. A morcellator may have an outer tubing which may comprise dents 911 and an/or an inner tubing which may comprise cut-outs 912.

In some examples, there may be substantially no relative movement between the camera module and the one or more instruments (e.g., disposable instruments). Such that the camera may be configured to provide a stationary view of the one or more instruments and/or tools. In some examples, the camera module and the one or more instruments (e.g., disposable instruments) may be permanently integrated to the endoscope, and there may be substantially no relative movement between the camera module and the disposable instrument.

In some examples, the camera module may be configured to provide a stationary view of the one or more disposable instruments. In some examples, the camera module may be fixed relative to the one or more disposable instruments.

In some cases, there may be, minimal or substantially no relative motion between the distal end of the morcellator and the camera. The combined device may provide a stationary or substantially stationary view of the morcellator in the camera vision. For example, the instrument (e.g., the morcellator) may not move relative to the camera module, and therefore be stationary in the picture the camera generates which may be helpful during procedures (e.g., observation or surgical procedures).

The location of the morcellator may be stable in the camera live view. Keeping the location of the morcellator stable in the camera live view may be advantageous for many reasons. For example, when operators try to move the combined device in subject's body. The combined device may simplify the ergonomics during the procedure. In some cases, the operators may only need to hold a single device comprising an endoscope (e.g., a single-use endoscope) which may have one or more instruments (e.g., other instruments) integrated therein instead of holding an endoscope and one or more additional instruments separately. Such instruments may comprise or be one or more camera(s), illumination apparatus, other instruments, and/or combinations thereof. The improved ergonomics may result from such integration which may greatly benefit and enhance the procedure workflow efficiency and procedure safety.

FIG. 9 shows various examples of an integrated device in substantially a side-by-side configuration. For instance, the example device 903 shows a variation of the side-by-side configuration. The device 903 may comprise a pre-bent morcellator 924 and an endoscope 926. As shown in the example device 903, the morcellator may have a pre-bent portion 928 around the distal end or close to the tip and the distal portion of the endoscope may locate at a neck area of the morcellator in proximity to the pre-bent portion 928. In some cases, the endoscope (e.g., endoscope 926) is not pre-bent and the tip of the morcellator (e.g., morcellator 924) may be captured in the camera view of the camera module 930 that is located at the distal portion of the endoscope. The pre-bent on morcellator may beneficially allow a better view of its tip in the camera's live vision.

In another example, such as in device 905, the endoscope 932 and the morcellator 934 can both have a pre-bent. The example device 904 shows a variation of the side-by-side configuration. The device 904 may comprise a pre-bent endoscope and a pre-bent morcellator. In this example, the pre-bent configurations may allow a better access to the patient body in places that may be hard to reach with a traditional rigid design as shown in the example device 901. The difference between example device 903 and example device 904 is that in device 903 the endoscope 926 is not pre-bent and only the morcellator 924 is pre-bent, while in device 904, both the morcellator 934 and the endoscope 932 comprise a pre-bent configuration.

Another variation of the side-by-side configuration is shown as example device 905. In the example device 905, the endoscope 936 may comprise an articulating and/or flexible shaft. For example, the elongate member of the endoscope may be an articulating or flexible shaft. In the example device 905, the instrument (e.g., morcellator 938) may also comprise an articulating and/or flexible shaft. The endoscope 936 and the instrument (e.g., morcellator 938) may each comprise a shaft which may be articulated with two or more degrees of freedom. In some cases, the camera 940 may reside at the distal end (such as the distal end of the endoscope 936). Alternatively, the camera may not reside at the distal end. Instead, the camera module can reside at other device locations, such as other areas described elsewhere herein. In such cases, there may be mechanical and/or electrical cables or illuminating fibers along the shaft. The shaft can be flexible and articulating. The morcellator (e.g., morcellator 938 or other instruments described in other devices herein) can be made by torque coil pipe which may be flexible (e.g., it may be made from a flexible material). This flexible design/configuration of the device (e.g., device 905) may allow improved or significantly improved accessibility to the patient body parts compared to existing devices (such as rigid devices).

Referring to FIG. 9, another example of the device is shown as device 902. The camera 908 location/position may be fixed relative to the morcellator (e.g., 911 and 912). Alternatively, the camera location and/position may be adjustable. Fixing the camera location may have several advantages as described anywhere herein. In some examples, the devices and systems provided herein may comprise an illumination source (or illumination parts) 909 which may be integrated in the device (any of the example devices, such as device 901 or 902), such as in certain device parts. In some examples, the illumination source may be incorporated close to or next to the camera or camera/imaging module 908 (such as in device 901 or 902).

The example device 902 may comprise a “scope-out circular” configuration. In some cases, there may be minimal or substantially no relative motion between the camera module and the morcellator distal end. Therefore, the camera module may be configured to provide a stationary view of the morcellator. For example, the instrument (e.g., morcellator) in the picture may not move in the picture provided through the camera module.

Similar to the side-by-side configuration, the scope-out circular configuration 902 can take the form of pre-bent. An example of this is shown in device 906.

Alternatively, an example device, such as device 907 may comprise a flexible articulating shaft. Both designs may improve the control of the device tip and accessibility to the patient body parts.

In some examples, the designs described herein may be altered or various combinations of the designs may be used to facilitate desired applications

Integrated Device with Endoscope and Inflatable Tip

In some examples, the one or more disposable instruments may comprise an inflatable tip. In some examples, the inflatable tip may comprise an expandable balloon. In some examples, the inflatable tip may be a structural member of a balloon. In some examples, at least one of the one or more disposable instruments may comprise a shaft and a distal portion of the shaft may be connected to the distal end. In some examples, the inflatable tip may comprise at least one of a structural tube and an internal stiffener. In some examples, the device may be configured to provide a stationary view of the inflatable tip. In some examples, there is substantially no relative movement between the inflatable tip and the camera module.

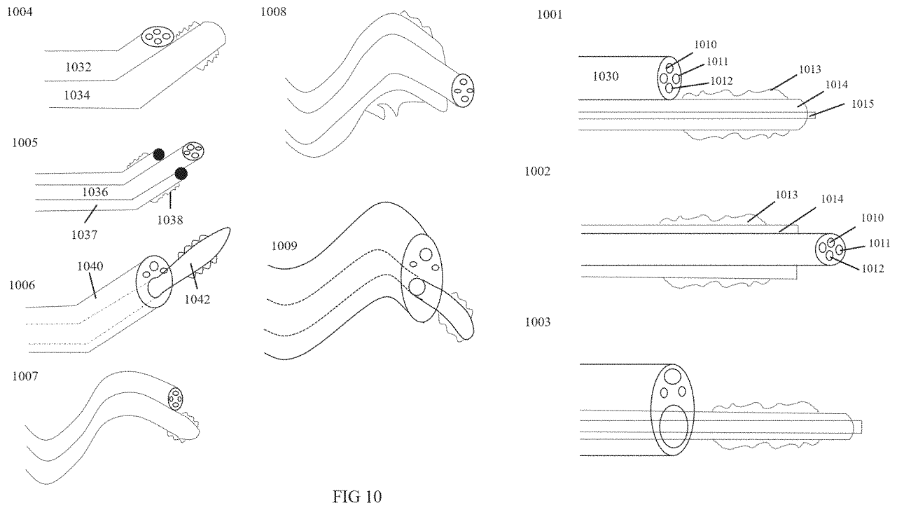

In some examples, the one or more instruments (e.g., the one or more disposable instruments) may comprise an inflatable tip, such as a balloon (e.g., an expandable balloon). Some examples of such devices are shown in FIG. 10.

Referring to FIG. 10, in some examples, such as device 1001 or the other devices provided herein, a camera module (e.g., a low-cost and/or disposable camera) 1010 can reside next to the inflatable tip (e.g., balloon) 1013 at the distal end (or other locations) of the device or of the endoscope (e.g., endoscope 1030) to form a side-by-side inflatable tip (e.g., balloon) with vision capabilities. In some cases, the device (e.g., device 1001) may further comprise an illumination source (or illuminating parts) (e.g., illumination source 1011) which may optionally reside next to the camera (e.g., camera 1010) or at other locations and/or positions on the device.

Optionally, the device may further comprise a channel 1012. For example, the endoscope 1030 may comprise a channel 1012. In some cases, the channel 1212 may be referred to as the working channel. The inflatable tip 1013 (e.g., balloon) may be expandable and/or may comprise a structural tube 1014 and/or an internal stiffener 1015. In some examples, there may be minimal or substantially no relative motions between the distal end of the inflatable tip 1013 and the camera 1010 (for example when the device is in use). The combined device (e.g., device 1001) may provide a fixed view of the balloon 1013 in the camera vision. The location of the balloon may be stable in the camera's live view. This may provide an advantage when operators try to move the combined device in patient body. In some cases, the combined device (e.g., device 1001 or other example devices provided herein) may simplify the ergonomics during the procedure. In some applications operators may only need to hold one device instead of a separate endoscope and one or more separate instrument(s). The improved ergonomics can improve the procedure workflow efficiency and procedure safety.

Referring to FIG. 10, the side-by-side configuration of the device (e.g., combined device comprising an endoscope and one or more instruments such as an inflatable tip) may comprise a pre-bent. An example may be device 1004. The pre-bent may be incorporated at the neck area of the device 1004 or other areas thereof. Example device 1004 comprises an endoscope 1032 and an instrument, which may be an inflatable tip 1034. In this example, both the endoscope 1032 and the inflatable tip 1034 may each comprise a pre-bent.

Alternatively or in addition, the device can be flexible (or fully flexible) and/or with distal articulation. In this case, driving cables or pull wires may be put in place in order to drive the tip of the combined device. Along the shaft of the device may be mainly composed of electrical and/or mechanical wires and optional illuminating fibers. In some cases, the inflatable tip (or balloon structure tube) 1014 can be soft and/or compliant.

As another example, a scope-in circular configuration (device 1002) is shown in FIG. 10. A camera 1010 may be incorporated in, integrated with, or built in the design. In some cases, the camera 1010 may be in the center of the device 1002. In device 1002, the camera 1010, illuminating parts 1011, and one or more optional channel(s) 1012 may be at the center of the combined device 1002. This configuration may be compact and relatively small in size. An inflatable tip (e.g., expandable balloon structural member) 1014 may exist next to the central bundle of the device. The inflatable tip or expandable balloon 1013 may be attached to the structural member 1014. The advantage of this configuration may be in that there may be no view of the inflatable tip (or balloon) in the camera. The reason for this may be that, in device 1002, the relative position of the camera module 1010 and the inflatable tip 10104 and/or balloon 1013 may be fixed. The inflatable tip 1014 or expandable balloon 1013 may be within the designed distance proximal to the camera 1010. The operator may have a clear view of the anatomy, and based on other indicators, i.e. proximal marks on the device, the operator may know where the inflatable tip or expandable balloon is relative to the camera view.

Another example of the scope-in circular design can be a pre-bent device shown in device 1005. Device 1005 may comprise an endoscope 1036 and an inflatable tip 1037 comprising a balloon 1038.

Alternatively, the balloon can be designed to be flexible and/or articulating as shown in device 1006. Device 1006 comprises an endoscope 1040 and a flexible and/or articulating balloon.

One other example is the scope-out circular configuration (device 1003). This configuration can have a pre-bent shaft (such as shown in 1006) or a flexible shaft (such as the one shown in 1009).

The side by side configuration and the scope-out circular design may have the advantage of direct visualization. Operators may be able to directly see and locate the balloon in the view while using the device. This feature may facilitate improved visualization and improved patient safety.

Integrated Device with Endoscope and Implant Delivery Capabilities

In some examples, the one or more disposable instruments may comprise an implant/stent delivery device. In some examples, the implant/stent delivery device may be configured to perform sinus procedures. In some embodiments, the device may comprise an implant/stent delivery device and an illumination source. In some examples, the device may further comprise a channel. In some examples, the device may further comprise an implant/stent delivery pushing mechanism. In some examples, the implant delivery device may be configured to deliver a prostate implant.

The one or more instruments (e.g., disposable instruments) may comprise or be an implant/stent delivery device or apparatus. A wide variety of implant types can be delivered to subjects or parts of subjects using a special design device endoscopically. A few examples of such implants are described herein. As an example, the implant delivery portion of the combined device may have different forms than the examples given. All design examples described below can be applied to all other implants and delivery devices.

### Sinus Implant Delivery

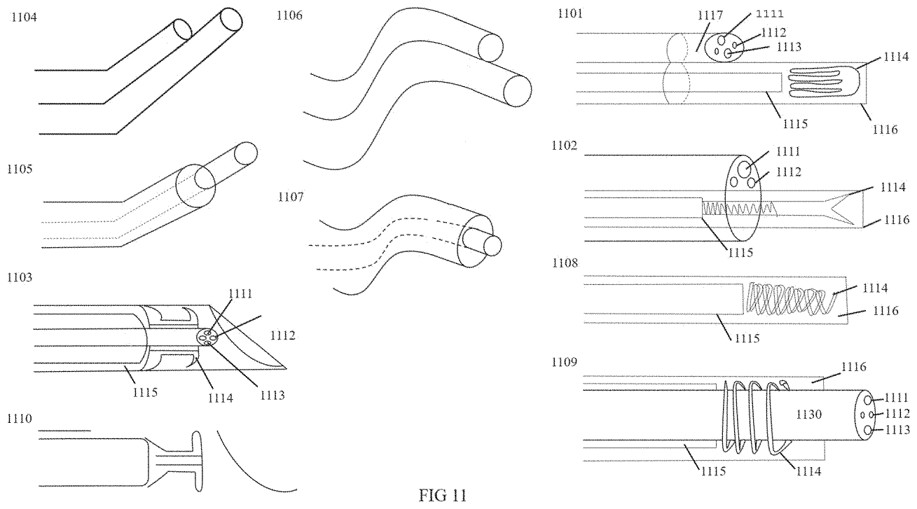

Referring to FIG. 11, an example device (e.g device 1101) may comprise a side-by-side configuration with endoscope 1117 and an implant delivery instrument (e.g., implant delivery tube) 1118 for performing sinus procedure. One or more camera(s) 1111 may reside at the distal end of the endoscope (e.g., endoscope 1117 in device 1101) and may stay fixed relative to the distal end of the implant delivery tube 1116. The implant delivery tube 1116 is an example instrument (e.g., disposable instrument) which may be integrated with an endoscope to make a device.

Alternatively, in some cases, the location and/or position of the cameras may not be fixed. In some cases, the location and/or position of the one or more cameras may be adjustable. In some examples, the device may further comprise an illumination source (or illuminating parts) (e.g., illumination source 1112 in device 1101). In some examples, the one or more instruments (e.g., the one or more disposable instruments) may further comprise an illumination source. The implant delivery instrument/tube 1116 may be disposable or single-use. The example device 1101 may be disposable. The illumination source 1112 may be disposable and/or single-use. The implant delivery instrument/tube 1116 and the illumination source may be integrated with the endoscope 1117 (e.g., a disposable endoscope) to make the device 1101 as a single unit. In some cases, the device 1101 may be disposable. In some cases, the device 1101 may comprise some pieces and/or elements which may not be disposable.

Referring to FIG. 10, the device (e.g., device 1101) may optionally comprise one or more channel(s) 1113. The system/device may comprise an implant 1114 which may sit inside of the implant delivery instrument/tube 1116 or at other locations on the device. The device may further comprise an implant delivery pushing mechanism 1115 which may reside at the proximal of the implant or other locations, and when activated, the pushing mechanism 1115 may push the implant 1114 out at the desired location of the sinus or any other intended body part. The implant delivery instrument 1116 of the device 1101 may be a sinus delivery instrument.

Referring to FIG. 10, another example of the device may comprise or be a scope-out circular configuration (e.g., device 1102). In this case, the device (e.g., device 1102) may be configured to deliver a different implant 1114. The camera module 1111 may reside at the distal end (such as the distal end of the endoscope) and may be fixed relative to the distal end of the outer structure of the implant delivery instrument 1116.

### Prostate Implant Delivery

Referring to FIG. 10, an example device can be designed as shown in example device 1108. This device 1108 may comprise various applications and uses comprising for example, delivering implant to a subject's body. In some examples, the device may comprise application(s) in delivering a prostate implant to a patient's body or body parts. This drawing shows the implant delivery portion/instrument of the device. The implant delivery instrument of the device 1108 may be a prostate delivery instrument. The device may comprise additional parts and pieces.

The device 1108 (e.g., a combined or integrated device) can have a side-by-side configuration or a scope-out circular configuration (such as according to the examples and FIGs described and shown anywhere herein).

Another example is illustrated as device 1109 in FIG. 11. The device 1109 may comprise an endoscope 1130, an implant delivery instrument 1116, an implant pushing mechanism 1115, a camera module 1111 at the distal tip of the endoscope 1130, an illumination source 1112, an optional 1113, and optional other parts and/or pieces. The device may be configured to deliver an implant 1114. In the example device 1109, the camera 1111 and illumination parts 1112 may pass through the center opening of the implant 1114, the implant pushing mechanism 1115 of device 1109, and the outer structure of the implant delivery instrument 1116 of device 1109. This configuration may be compact in size. The device 1114 may be configured to or capable of performing the functions of both the implant delivery instrument and the endoscope. In some cases, the entire device may be disposable. Compact designs may comprise various advantages for various purposes, such as intended surgical or examination procedures.

### Ear Tube/Implant Delivery

In some examples, ear tubes, comprising for example, tympanostomy tubes, ventilation tubes, pressure equalization tubes, and/or other types of tubes for various applications can be delivered using a device provided herein.

Referring to FIG. 11, one example of the tube delivery device is illustrated as the example device 1110. This delivery portion can be part of the side by side configuration or the scope-out circular configuration by replacing the implant delivery portion as shown in 1101 and 1102.

Referring to FIG. 11, a scope-in circular configuration is illustrated as the example device 1103. This design may be similar to the one shown in 1109. In some cases, the side-by-side designs shown in FIG. 11 can have a pre-bent portion which may be located at the neck area of the device or other locations, such as shown in 1104. A flexible (e.g., fully flexible) design with articulating is shown in 1106. The scope-in circular design and the scope-out circular design can have a pre-bent, such as shown in 1105. Alternatively or in addition, they may have a flexible design, such as shown in FIG. 1 (107). Other examples of flexible and articulating devices are illustrated in FIGS. 9 and 10. Such configurations may be combined with the implant delivery devices illustrated in FIG. 11, and various designs and configurations may be provided.

Integrated Device with Endoscope and Stitching Capabilities

In some examples, the one or more instruments (e.g., the one or more disposable instruments) may comprise or be stitching devices (or mechanisms), such as devices which may have stitching capabilities and may be configured to perform stitching. Stitching mechanisms may be disposable. Stitching mechanisms may be integrated in the device to make a single unit. The integrated unit comprising an endoscope and a stitching device or mechanism may be disposable.

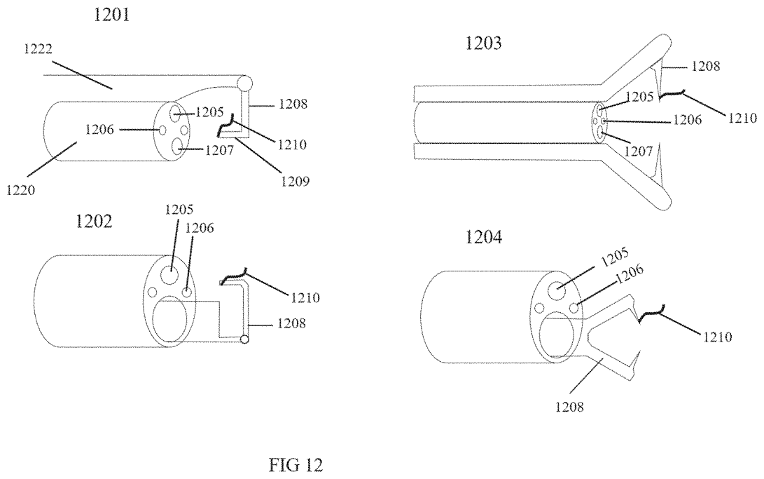

Referring to FIG. 12, an example device 1201 with the side-by-side configuration is illustrated. The device 1201 may comprise an endoscope 1220 and a stitching instrument/mechanism 1208 A camera module 1205 may reside at the distal end of the endoscope 1220 and may sit next to the stitching mechanism 1208. In some cases, the location of the camera may be fixed on the device (for instance relative to the stitching mechanism). The device/system 1201 may further comprise one or more illumination sources (or illuminating parts) 1206. Optionally, the device may further comprise one or more channels 1207. The stitching mechanism 1208 may be customized and may comprise one or more stitching needle(s) 1209. A suture 1210 may be attached to the stitching needle 1209.

Another example may comprise the scope-out configuration shown in example devices 1202 and 1204. Stitching mechanisms 1208 may exist at the distal of the endoscope (e.g., next to the camera). In some cases, the camera module 1205 and the one or more instruments (such as the stitching mechanism 1208) may both reside at the distal end (such as at the distal end of the endoscope). Additional alternative designs and configurations may be possible.

Another example is illustrated as device 1203 which comprises a scope-in circular configuration. In device 1204, the camera module 1205, illumination source (or illuminating parts) 1206, and the channel 1207 may be located at the central location of the device. The device can have rigid shaft, pre-bent shaft, or flexible articulating shaft. Designs and configurations of the pre-bent shafts and the articulating shafts may be similar to the pre-bent or articulating shafts provided anywhere herein, such as in FIGS. 9 to 11 or elsewhere herein.

Integrated Device with Endoscope and Basic Instruments

In some examples, the devices provided herein may comprise an endoscope and one or more basic instruments. The endoscope may be disposable. The one or more basic instruments may be disposable. The device may be comprised of integration of the endoscope and the one or more instruments (e.g., the one or more basic instruments which may be disposable). Therefore, in some cases, the device may be disposable and single-use. The device may be configured to perform the functions of the endoscope and the one or more basic instruments.

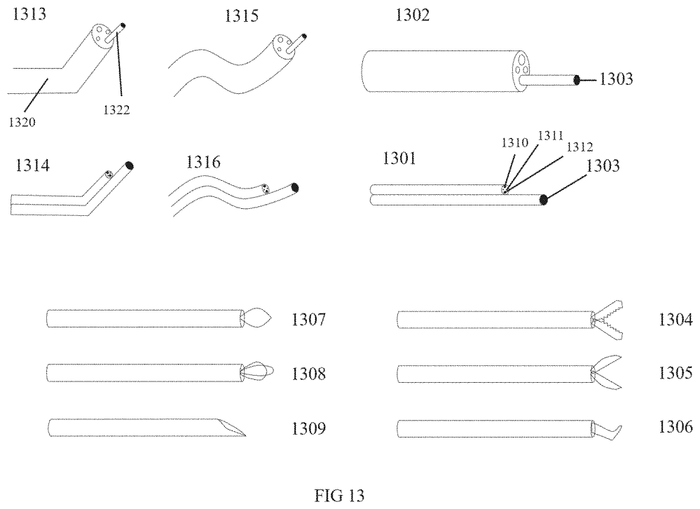

Referring to FIG. 13, in some examples, other tools and/or instruments may be used along with endoscopes for certain procedures and/or applications. Examples of such instruments may comprise laser fibers (e.g., shown in device 1303), graspers (e.g., in device 1304), scissors (e.g., in device 1305), snares (e.g., in device 1306), loops (e.g., in device 1307), buckets (e.g., in device 1308), needles (e.g., in device 1309), other instruments and/or any combinations thereof.

For example, device 1313 comprises an endoscope 1320 and laser fibers 1322. Laser fibers (instrument) may be integrated with the endoscope as a single integrated device which in some cases, may be disposable. The device may comprise any configuration, such as rigid, flexible/articulating or pre-bent. In the example device 1313 illustrated herein, the endoscope comprises a pre-bent configuration. In other examples, any of the instruments listed herein may be integrated with endoscopes in any configuration (e.g., any configuration provided herein).

One example of the side-by-side combined device is illustrated as device 1301. The camera 1310 may sit distally (such as at the distal end) and may be at a fixed location relative to the fiber tip 1303.

A scope-out circular configuration is shown in example device 1302. The device may comprise a fiber tip 1303 or laser fiber. Alternatively, the fiber tip may be replaced/exchanged with any other instrument, such as the instrument illustrated in example instrument 1304-1309 in FIG. 13.

Referring to FIG. 11, in some examples, the device may comprise a pre-bent 1313 and 1314. A flexible configuration with articulation is shown in example devices 1315 and 1316. A cable driving mechanism (not shown) may reside inside the device (such as inside the elongate member of the endoscope or in the instrument or inside the shaft of endoscope and/or instrument as desired) in order to achieve the articulation and flexibility for various purposes.

### Integrated Device Proximal End and Supporting Member

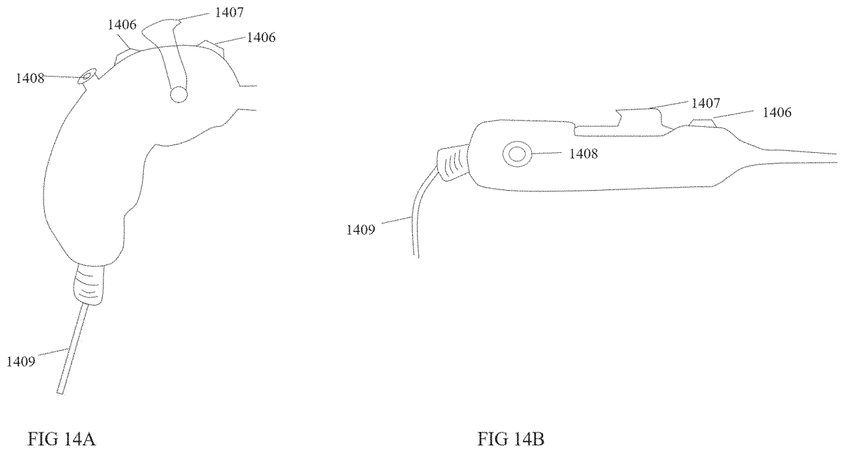

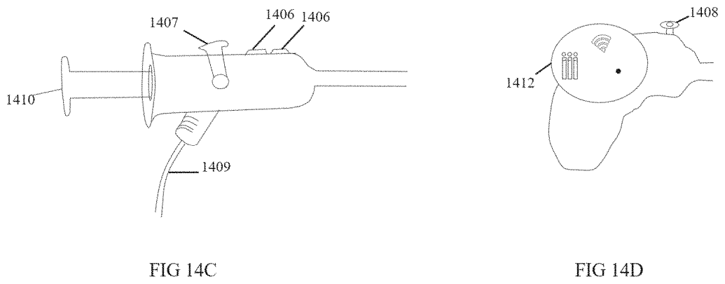

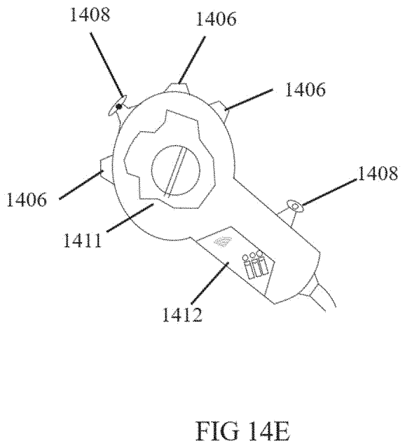

Examples of device are shown in FIGS. 14A to 14E.

One example with ergonomic grip is shown in FIG. 14A. The device may comprise buttons 1406 (for example to control the camera module, illumination source, and/or other instruments). The device shown in 14A may comprise a lever or sliding bar 1407 which may facilitate controlling the articulation of the device (e.g., the combined device) at a given location (e.g., at its distal end). A fluid lure 1408 may be present to allow fluid inflow and outflow. The proximal end of the device may be attached (in some cases removably) to a supporting member. The proximal end of an example device is shown in FIG. 8. An example supporting member (a hand-held device) connected to a proximal end of an example device is shown in FIG. 3.

In some examples, the device may comprise or be connected to a hand-held device or a hand piece (such as the hand-held device shown in FIG. 3). An electrical cable 1409 may be located at the proximal end of the hand piece and may connect to an example display system.

The display system may be any kind of display system such as a computer, a computational device, a laptop, a PC, an iphone, an ipad, a tablet, a monitor, a screen, an LCD, or any other display device or system. In some cases, multiple display device or systems may be used.

A straight grip example of the hand-held device (or hand piece) is shown in FIG. 14B. The lever or sliding bar 1407 (or another mechanism) may be used to control the articulation of the combined device at distal end.

Another example of the hand-held device (or hand piece) is shown in 14E. In some examples, the device may comprise a knob 1411 (such as a rotating knob) which may be used to control the articulation of the device (e.g., combined or integrated device). For the integrated implant delivery device (a device comprising an endoscope and an implant delivering device, such as any such device provided anywhere herein), a push bar 1410 may be available at the proximal end of the hand-held device (or hand piece) which may allow for the delivery of the implant.

Another example of transmitting the power and image may be through wireless. A wireless module pack 1412 is shown in 14E and 14D. The pack may comprise a wireless emitter and/or a plurality of batteries.

Provided herein are systems and methods for addressing medical conditions (such as gynecological conditions or other conditions) in subjects, such as animal and human subjects, and/or parts of subjects, such as any body part, organ, or tissue of any subject. The method and systems provided herein may comprise applications in various domains spanning various conditions (healthy or diseases) in various parts of subjects (e.g., different body parts). Such applications may comprise gynecological applications, polypectomy, myomectomy, Intrauterine device (IUD) delivery, uterus cancer treatment and other applications. For example, subject parts to be addressed, diagnosed, observed, examined, or treated may comprise sinuses, bladder, colon, esophagus, blood vessels, throat, brain, heart, lungs, intestines, eyes, skin, liver, pancreas, stomach, kidney, ovaries, testicles, bladder, ear, nose, mouth, bone marrow, adipose tissue, muscle, glandular, mucosal tissues, spinal, nerve tissues, cartilage, teeth, and bone.

In some examples, the present disclosure provides devices, such as disposable or single-use devices with integrated vision capabilities (such as one or more camera modules) to treat polypectomy, myeomectomy, IUD (Intrauterine device) delivery, and other conditions.

The devices provided herein may comprise endoscopes. In some cases, the endoscopes provided herein may be disposable or single-use which may provide several advantages over re-usable endoscopes, as discussed anywhere herein. In some examples, the devices and systems may further comprise other instruments which may be integrated with them. Alternatively, such instruments may be used separately but in combination with the endoscopes. Integrating such instruments with the endoscopes (e.g., single-use endoscopes) may provide numerous advantages as described anywhere herein.

The devices and systems may be used to address, diagnose, and/or treat various conditions in subjects or parts of subjects. Subjects may comprise animals, mammals, rodents, and human such as patients. Conditions may comprise healthy or diseases conditions. For example, the devices and/or systems may be used to for diagnosis and/or treatment of polypectomy and/or myomectomy. The devices or systems may be used for intrauterine device (IUD) delivery. The devices and systems may be used for various applications.

Polyps, fibroids, may be considered common diseases among women, especially at older ages. In some cases, these diseases may involve and/or be associated with having abnormal uterus bleeding. An intrauterine device (IUD) is an approach for birth control, widely used by women. Minimally invasive surgical treatments to polyps (polypectomy) and fibroids (myomectomy) are often used in practice. Same type of surgical procedures may be available to place IUDs in patients' bodies. Such methods may comprise mechanical removal (polyps, fibroids) or placement (IUD), electrical cautery devices for polyps or fibroids, morcellators for removing polyps or fibroids, and more. In many cases, a hysteroscope or resectoscope may be used to access to the uterus. These endoscopes may be comprised of multiple parts including sheath, telescope, tool channel and water channels. In many cases, they may require assembly of several or all pieces together before example surgical procedures. In some cases, the treatment tool or instrument may be inserted into a device channel, such as an endoscope channel. For example, a morcellator may get inserted into the endoscope channel to do polypectomy or myomectomy. Morcellators may comprise at least one myosure device, truclear device, and/or other devices and/or instruments. However, it may be preferred to design devices which comprise both an endoscope and a morcellator as a single integrated unit. Provided herein are such devices. For example, a device may comprise an endoscope and one or more instruments. The instruments may comprise or be a morcellator. The endoscope and/or the one or more instruments may be disposable. The integrated device may be disposable.

In other scenarios, the one or more instruments (e.g., the one or more disposable instruments) can comprise or be electrical cautery devices, such as symphion device, TCRis, and/or other devices. The methods may comprise using uni-polar bi-polar energy to remove polys or fibroids or to treat other conditions. Other examples may comprise ablation devices for uterus, such as RF ablation, or other. In some examples, any of the instruments listed herein may be integrated with an endoscope to make a single device which may be partially or entirely disposable. In some examples, the instruments or tools may be disposable. In some examples, some instruments may be used which may not be disposable. Therefore, in some cases, some portions of or the entire device may be re-usable.

In some cases, the instrument can be laser-based, comprising using one or more types of lasers such as a green laser, a holmium laser, or other types of lasers. For example, the one or more instruments (e.g., the one or more disposable instruments may comprise or be a laser, such as the laser listed herein as examples). In some examples, a device, such as an IUD delivery instrument, such as a grasper, may be inserted into the channel of an endoscope to deliver the IUD. Such device may be integrated with the endoscope to make the device. The device may be integrated and disposable.

Various kinds of mechanical instruments can be used to mechanically remove polys or fibroids. Examples may comprise graspers, scissors, snares, baskets, needles, and more. As an example, needles can be used to deliver medication or drugs to specific locations. In other examples, needles may deliver steam or cryo therapies. The one or more instruments (e.g., the one or more disposable instruments) may comprise any of the instruments provided herein and/or any combinations thereof.

In some examples, instruments may be designed to be low-cost and/or single-use for sterility purposes. Conventional endoscopes can be expensive, sometimes due to precisely-made optics or for other reasons. Endoscopes are traditionally made as reusable pieces and may often be used as an equipment. Recognized herein is a need for developing single-use/disposable endoscopes for reasons described anywhere herein.

In some cases, prior to surgical procedures, physicians may to assemble the reusable endoscopes into single-use instruments/tools, or vice versa. After each procedure, there may be a need to dis-assemble the endoscopes from other instruments and clean and sterilize the endoscopes and/or other parts and/or instruments. Providing disposable and or single-use endoscopes may eliminate the need for such post-procedure cleanings. In addition, providing integrated devices comprise a disposable endoscope and one or more instruments (such as disposable instruments or tools) according to any of the examples provided herein may eliminate the need for user-assembly.

Traditionally, endoscopes may often comprise a channel (such as a working channel) which may allow the instruments to go through and operate under endoscopy views. Alternatively, instruments may have scope channels that may allow telescopes to be inserted into and instruments can operate under endoscopy views. With more commonly used single-use instruments, this combination may make a mixed scenario where endoscopes may still require cleaning and sterilization. To improve the clinical workflow and efficiency, a single-use package with single-use instrument and single-use endoscope is desirable. Such devices and systems are provided herein along with example methods of use.

When both the instrument and endoscope are disposable or single-use, it may be desirable to integrate them into one combined device. With a low-cost camera module, integration of such cameras onto single-use devices may be desirable to streamline the workflow of such devices. Integration of instruments with vision capabilities can optimize devices' overall size and decrease or minimize patient pain.

Traditionally, endoscopes may use a lens or a stack of many optical lenses. This may make it difficult to implement a flexible and soft endoscope. With small and low-cost camera modules, flexible and soft shafts may be desirable. It may improve patient comfort in many applications, such as polypectomy, myomectomy, IUD delivery, and more

An integrated single-use device with integrated instrument and vision capabilities may provide advantages such as sterility, lowered cost, smaller overall size, improved patient comfort, better usability, and simpler workflow among other advantages.

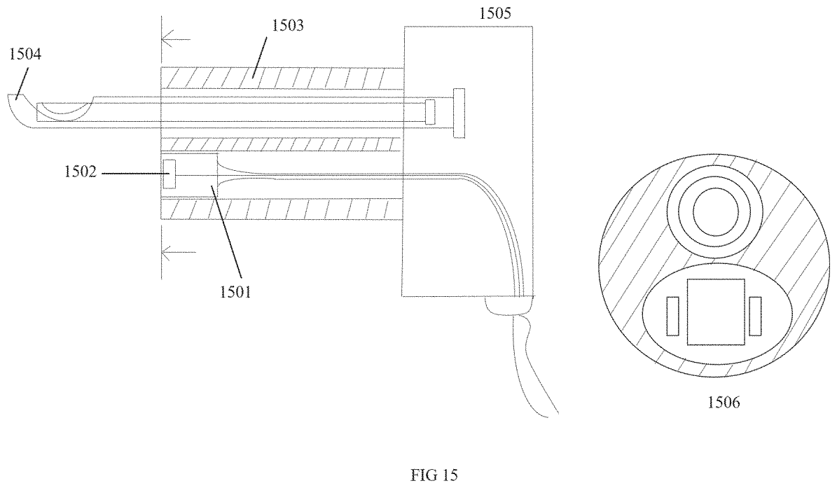

FIG. 15 shows one example of the distal portion of a single-use (or disposable) integrated device which may further comprise vision capabilities. An imaging module or camera module such as a camera sensor 1501 may reside at the distal of the device or other device locations of the device. In some cases, the camera sensor may comprise or be a low-cost camera sensor. The camera sensor may comprise a lens. For example, the camera sensor may comprise or be complementary metal-oxide-semiconductor (CMOS) or complementary-symmetry metal-oxide-semiconductor, or a charge-coupled device (CCD), and/or other devices or parts. Other camera modules and/or imaging devices may be used. In some examples, an integrated illumination device or part, such as a light source may be attached to the distal tip 1502 or be located at other device regions/area. The illumination device or light source may comprise or be one or more of a light-emitting diode LED, light fibers, lasers, ultraviolet light (UV light) source, or any illumination device or light source generating light with any wavelength and/or characteristics. Both the camera, camera sensor, or camera module and/or the light source may be attached to the device distal tip or other device parts, area, locations or zones at various positions. In some cases, such devices or parts may be permanently attached or located in the intended location or position. Alternatively, such devices or parts may be removably attached or connected to such device zones (e.g., the device distal tip or other locations on the device). In some cases, the camera module can be tilted with a given angle (e.g., inside the integrated device), which angle may be any angle, for example, 0, 12, 18, 30, 50, 60, or more. This may facilitate creating an endoscope with vision capabilities. Such vision capabilities may comprise front view, side view, view from a single angle, view from multiple angles, and more. In some cases, multiple cameras may be installed, attached or connected to the same or multiple locations on the devices at the same or at alternating angles (any angle). The location and/or position of each camera may be fixed or adjustable. In some examples, data such as images, videos, or views collected from or generated by one or more cameras attached to or installed on the device could be combined or otherwise processed or used to create a 360° view of the environment in which the device or system is inserted into or operating in.

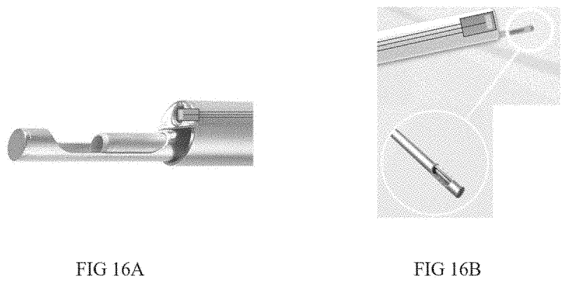

In some examples, the device may comprise a shaft. In some examples, the shaft may be a multi-lumen shaft. Inside the shaft (e.g., multi-lumen device shaft) 1503, one or more channel(s) may be used to allow a morcellating shaft 1504 to move at desired directions (for example, axially in or out of the tip). Alternative directions may comprise moving vertically or at any angle. In some examples, the morcellating shaft may rotate radially along the shaft. FIGS. 16A and 16B show two possible versions of the morcellating shaft. Morcellating shaft is provided as an example. In other examples, one or more instruments (e.g., disposable instruments), such as the instruments or tools provided anywhere herein may be used instead of the morcellator.

In some examples, the one or more channel(s) (e.g., the endoscope channel or working channel) may allow fluid to flow in or out of the subject's (e.g., patient) body. A side view of the cross-section of such example device is shown in 1506. The proximal end of the device is shown as a block in 1505. More details of the proximal end and the supporting member (e.g., hand-held device or hand piece) are shown in FIG. 3.

FIGS. 2A and 2B shows an alternative distal portion of a single-use integrated device which may further comprise vision capabilities (such as camera module). Similar to the arrangement in FIG. 5, the camera module and illumination source may be attached to the distal tip. Alternatively, in some examples, the camera module, the illumination source, and/or both may be located at other locations on the device or off device. In each case, such instruments, modules, and/or apparatus may be used in combination with the device (e.g., endoscope such as single-use endoscope provided herein). Referring to FIGS. 2A and 2B, next to the camera module (or imaging module), the device may comprise an electrical cautery delivery channel 201, an IUD delivery channel, or other one or more channels incorporated for the same and/or other functions or purposes. In other examples, such channels may be located/positioned in other locations, areas, or zones on the device. The delivery channel 201 may move relative to the shaft of the device axially. Alternatively, the delivery channel 201 may move vertically or radially relative to the shaft of the device. A cross-sectional view of the front face 202 is shown in FIG. 2B as an example.

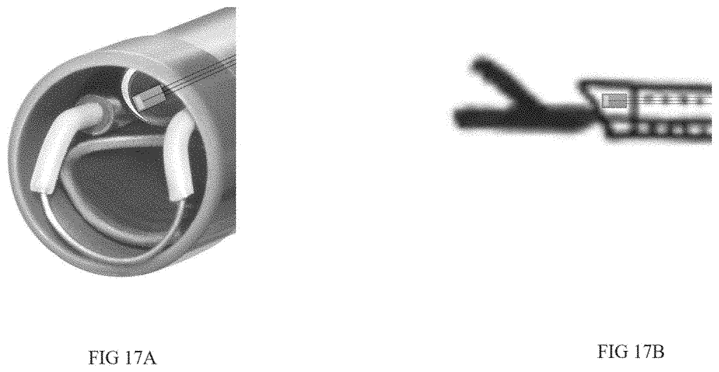

One example of a device (e.g., single-use or disposable device) according the systems and methods of the present disclosure is shown in FIG. 17A. In this example, an electrical cautery device is provided next to the camera module. In other example, one or more camera modules may be located in the same or other locations on the device. Another example of a single-use device is shown in FIG. 17B. Such as shown in FIG. 17B, one or more camera module(s) may be comprised in the device. In some cases, the camera module may be incorporated next to the mechanical grasper which may be used as part of the device to address a condition in a subject, such as to treat polyps, fibroids, deliver IUDs or other devices, or be used for other applications.

Referring to FIGS. 2A and 2B, in some examples, the channel 201 can hold mechanical, electrical, optical, or other energy devices, comprising, for example, graspers, scissors, snares, buckets, needles, uni-polar, bi-polar cautery instruments, radiofrequency (RF) ablation instrument, green laser fiber, holmium laser fiber, or other devices, apparatus, and/or instruments.

In other examples, the channel 201 can hold needles for biopsy, steam delivery, cryotherapy delivery, medication delivery, or other purposes. The channel 201 may further be capable of holding other instruments for other purposes.

The examples shown in FIG. 15 and FIGS. 2A and 2B, can comprise a rigid, semi-rigid, flexible, or articulatable device shaft. In each case, the implant delivery channel can be rigid, semi-rigid, flexible, or articulatable (e.g., articulating with two or more degrees of freedom).

One example of the proximal side of the devices is shown in FIG. 3 Two examples of an example supporting member (e.g., a hand-held device or hand-piece) are shown in FIGS. 6A and 6B. Inside the hand piece, as an example, the IUD delivery channel can have stops (e.g., hard stops) 301 along axial direction. This may provide safety stops to avoid excessive insertion of the channel into subject's body.

The device may comprise mechanical or electrical interfaces such as mechanical or electrical buttons. Buttons (such as mechanical and/or electrical buttons) 302 may reside on the proximal side or other locations on the device and may enable insertion of morcellator shaft for example. Other uses of the buttons may comprise opening of mechanical grasper jaws or load IUD cartridges. Other buttons may allow image capture, video recording, white balance, or other applications and/or functions.

In some cases, mechanisms such as internal mechanisms may be in place for connecting buttons (e.g., mechanical buttons) to the morcellator shaft insertion or grasper jaws. An example may comprise or be a hinge 305 which may connect the button (or buttons) 302 to the channel 300. This mechanism can comprise other forms which may be more complicated. Mechanisms may comprise mechanical and/or electrical mechanisms. In some examples, one or more interfaces 303 for fluid management may be provided at proximal end of the device. Such interfaces may be lure connectors to enable inflow and/or outflow of fluid, or other media.

The one or more interfaces may allow IUD delivery instruments or other instruments, such as the instruments mentioned anywhere herein or other instruments to go through. Another use of the one or more interfaces may comprise allowing a separate single-use endoscope to be inserted and/or snapped to the rest of the device, for example, in a case where there is no integrated camera module at distal tip of the device. Alternatively, in some cases, the one or more interfaces may comprise allowing a separate single-use endoscope to be inserted and/or snapped to the rest of the device when the device does comprise a camera module at its distal tip or other locations.

In some examples, a mechanical and/or electrical interface 304 may be present to allow the device to connect to an image processing and/or display system, which may comprise or be a laptop PC (such as a laptop PC with a display monitor) or another device. Alternative options may comprise imaging processing unit and/or display monitor.

Other ways to implement the device (such as integrated device) with vision capabilities may comprise an outer shaft with electrical cautery device and may further comprise a camera module and/or illumination device/module/apparatus/part within the outer shaft or at other locations. FIG. 16A shows one example of such design.

For design simplicity or adaptability reasons, the disposable (or single-use) camera module with illumination can be on a separate shaft, which can get inserted into a channel (for example inside the device shaft) or at other locations or positions. Once inserted, the camera module shaft can be snapped onto the device shaft which may make it one integrated device. In this example, the camera module shaft may be removed from the integrated device. Other designs and/or configurations may also be possible.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device comprising:
(a) an endoscope comprising: i) a disposable elongate member comprising a proximal end and a distal end and ii) a camera module located at the distal end, wherein the proximal end is removably attached to a supporting member; and
(b) one or more disposable instruments integrated to the endoscope,
wherein the device is configured to perform functions of both the endoscope and the one or more disposable instruments.

2. The device of claim 1, wherein the distal end is articulated in two or more degrees of freedom.

3. The device of claim 1, wherein at least one of the one or more disposable instruments is integrated to the endoscope through a permanent connection at the distal end.

4. The device of claim 1, wherein the one or more disposable instruments are one or more instruments selected from the group consisting of a morcellator, scissors, a needle, an illumination source, a laser, a balloon, an implant/stent delivery device, a stitching device, laser fibers, a grasper, snares, a loop, and a bucket.

5. The device of claim 1, wherein the camera module and the one or more disposable instruments are permanently integrated to the endoscope, and wherein there is substantially no relative movement between the camera module and the one or more disposable instruments.

6. The device of any one of claim 1, wherein the camera module is configured to provide a stationary view of the one or more disposable instruments.

7. The device of claim 1, wherein the camera module is fixed relative to the one or more disposable instruments.

8. The device of claim 1, wherein the supporting member comprises a mechanical interface configured to control an articulation of the distal end of the endoscope or operations of the one or more disposable instruments.

9. The device of claim 8, wherein the mechanical interface comprises a driving mechanism for one or more pull wires attached to the distal end.

10. The device of claim 8, wherein the mechanical interface comprises a mechanism for manipulating the one or more disposable instruments.

11. The device of claim 1, wherein the supporting member is a robotic arm or is coupled to a robotic arm and is configured to control functions of the endoscope and the one or more disposable instruments.

12. The device of claim 1, wherein the supporting member is a hand-held device.

13. The device of claim 1, wherein the one or more disposable instruments comprise a morcellator.

14. The device of claim 13, wherein the device is configured to provide a stationary view of the morcellator.

15. The device of claim 13, wherein the morcellator is pre-bent.

16. The device of claim 13, wherein the camera module resides at the neck area of the morcellator.

17. The device of claim 13, wherein the camera module and the morcellator comprise a shaft.

18. The device of claim 17, wherein the shaft is articulated in two or more degrees of freedom.

19. The device of claim 1, wherein the one or more disposable instruments are integrated to the endoscope using an adhesive material.

20. The device of claim 19, wherein the adhesive material is biocompatible.
